(19) United States
(12) Patent Application Publication
(10) Pub. No.: US 2020/0062776 A1
(43) Pub. Date: Feb. 27, 2020

(54) HEDGEHOG ACYLTRANSFERASE INHIBITORS AND USES THEREOF

(71) Applicants: Memorial Sloan-Kettering Cancer Center, New York, NY (US); Tri-lnstitutional Therapeutics Discovery Institute, Inc., New York, NY (US)

(72) Inventors: Marilyn D. Resh, New York, NY (US); James John Asciolla, New York, NY (US); Brittany Haugen, New York, NY (US); Masanori Kawasaki, Fujisawa (JP); Tomoya Yukawa, Yokohama, Kanagawa (JP); Kazuyoshi Aso, New York, NY (US); Michael Andrew, Foley, NY (US)

(73) Assignees: Memorial Sloan-Kettering Cancer Center, New York, NY (US); Tri-lnstitutional Therapeutics Discovery Institute, Inc., New York, NY (US)

(21) Appl. No.: 16/310,356

(22) PCT Filed: Jun. 16, 2017
(86) PCT No.: PCT/US2017/037843

§ 371 Date: Dec. 14, 2018

Related U.S. Application Data
(60) Provisional application No. 62/351,176, filed on Jun. 16, 2016.

(51) Int. Cl. C07D 495/04; C07D 519/00

(52) U.S. Cl. CPC C07D 495/04; A61K 45/06; C07D 519/00

(57) ABSTRACT

Hedgehog acyltransferase (Hhat), a membrane-bound O-acyl transferase (MBOAT) protein, is responsible for the palmitoylation of Shh and is crucial to proper Shh signaling. Hhat inhibitors that are capable of preventing Shh palmitoylation and mitigating Shh signaling, and therefore can be used in the treatment and/or prevention of diseases (e.g., proliferative diseases, such as cancer). Provided herein are Hhat inhibitors, such as compounds of Formula (I), (II), and (III), which are useful for the treatment and/or prevention of disease.

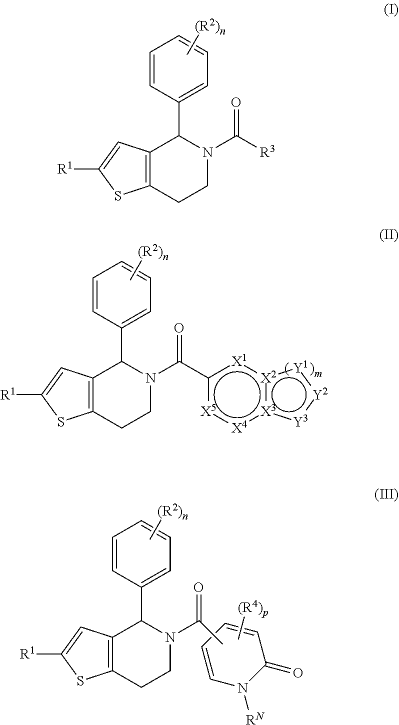

### RELATED APPLICATIONS

This application claims priority under 35 U.S.C. § 119(e) to U.S. Provisional Patent Application, U.S. Ser. No. 62/351,176, filed Jun. 16, 2016; the entire content of which is incorporated herein by reference.

### GOVERNMENT SUPPORT

The present invention was made with government support under grant numbers R21 CA186957 and RO1 GM116860, awarded by the National Institutes of Health. The government has certain rights in the invention.

### BACKGROUND OF THE INVENTION

Cancer is a disease for which there remains a great unmet medical need. According to the World Health Organization, about 14.1 million new cases of cancer were diagnosed across the globe in 2012, resulting in approximately 15% of human deaths that year. In particular, pancreatic cancer alone resulted in approximately 330,000 deaths worldwide in 2012 (see, e.g., World Cancer Report 2014, World Health Organization). Pancreatic cancer is the fourth most common cause of death from cancer in the United States, and only 25% of those diagnosed survive one year after diagnosis. The top three causes of death from cancer in the United States vary by gender, and include breast cancer for women and prostate cancer for men (see, e.g., Lifetime Risk of Developing or Dying From Cancer. American Cancer Society, Oct. 1, 2014; Cancer Facts & Figures. 2010. American Cancer Society, 2010).

The Hedgehog signaling pathway is responsible for delivering information to embryonic cells in order to promote proper cell development. The Hedgehog (Hh) signaling protein is a ligand found primarily in fruit flies, particularly in fruit flies belonging to the genus Drosophilia. Mammals express three closely related Hedgehog family members, including Sonic Hedgehog (Shh), Indian Hedgehog (Ihh), and Desert Hedgehog (Dhh). Sonic Hedgehog (Shh) plays a critical role in the development of embryonic cells; however, Shh expression is turned off in most post-embryonic cells. It has been found that in adult tissue, aberrant Shh signaling is linked to the development of proliferative diseases such as cancer. For example, adult pancreatic cells normally do not express Shh, and aberrant Shh expression has been found to promote the development and/or propagation of pancreatic cancer (see, e.g., Morton et al. Cell Cycle 2007, 6, 1553-1557). In fact, it has been demonstrated that inhibition of Shh signaling is effective against pancreatic cancer in mouse models (see, e.g., Olive et al. Science 2009, 324, 1457-1461; Feldman et al. Cancer Research 2007, 67, 2187-2196).

Hedgehog acyltransferase (Hhat), a membrane-bound O-acyl transferase (MBOAT) protein, is responsible for post-translational modification (e.g., palmitoylation) of Hedgehog proteins (e.g., Dhh, Ihh, Shh) and is therefore crucial to proper Hedgehog signaling (e.g., Shh signaling). In particular, Hhat inhibitors that are capable of preventing Shh palmitoylation and mitigating Shh signaling are promising agents for the treatment of diseases (e.g., proliferative diseases such as cancer and inflammatory diseases). Hedgehog acyltransferase is also suspected as being involved in non-canonical pathways, and therefore other signaling pathways could be affected by Hhat inhibition. Small molecule inhibitors of Hhat have been developed for the treatment of diseases, including proliferative and inflammatory diseases. For examples of 5-acyl-6,7-dihydrothieno[3,2-c]pyridine inhibitors of Hhat, see International Publication No. WO 2013/142253, published Sep. 26, 2013, which is incorporated herein by reference.

### SUMMARY OF THE INVENTION

In mammals, Hedgehog signaling pathway (e.g., Shh signaling pathway) is linked to the development of proliferative diseases (e.g., inflammatory diseases, cancer). Hedgehog proteins, including Sonic hedgehog, undergo post-translational modifications that are critically important to their signaling capabilities, including the ligation of fatty acids, such as palmitate. Hedgehog acyltransferase (Hhat) is responsible for the palmitoylation of Hedgehog proteins (e.g., Shh), and is therefore crucial to proper Hedgehog signaling (e.g., Shh signaling). Hhat inhibition can prevent post-translational modification of Hedgehog proteins and mitigate Hedgehog signaling, and are therefore promising agents for the treatment of diseases associated with aberrant Hedgehog signaling (e.g., proliferative diseases such as cancer and inflammatory diseases). For example, small molecule inhibitors of Hhat can prevent Shh palmitoylation and mitigate Shh signaling, and are therefore agents for the treatment of diseases associated with Shh signaling (e.g., proliferative diseases such as cancer and inflammatory diseases). Provided herein are Hhat inhibitors, such as compounds of Formulae (I), (II), and (III), which are useful for the treatment and/or prevention of diseases, such proliferative diseases. Exemplary proliferative diseases include, but are not limited to, cancers, benign neoplasms, diseases associated with angiogenesis, inflammatory diseases, and autoimmune diseases.

In one aspect, the present invention provides compounds of Formula (I):

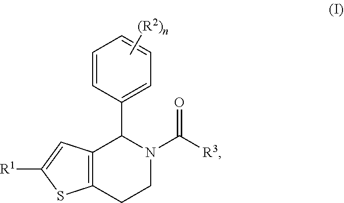

and salts, hydrates, solvates, polymorphs, co-crystals, tautomers, stereoisomers, isotopically labeled derivatives, and prodrugs thereof, wherein R¹, R², R³, and n are as defined herein.

In another aspect, the present invention provides compounds of Formula (II):

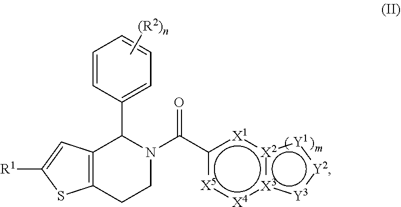

and salts, hydrates, solvates, polymorphs, co-crystals, tautomers, stereoisomers, isotopically labeled derivatives, and prodrugs thereof, wherein R¹, R², X¹, X², X³, X⁴, X⁵, Y¹, Y², Y³, m, and n are as defined herein.

For example, in certain embodiments, a compound of Formula (I) or Formula (II) is of the following formula:

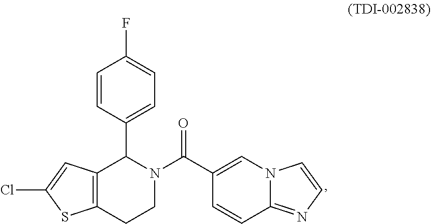 [SMILES: O=C(c1ccc2nccn2c1)N1CCc2sc(Cl)cc2C1c1ccc(F)cc1]

or a pharmaceutically acceptable salt, hydrate, solvate, polymorph, co-crystal, tautomer, stereoisomer, isotopically labeled derivative, or prodrug thereof.

Exemplary compounds of Formula (I) and Formula (II) include, but are not limited to, the following:

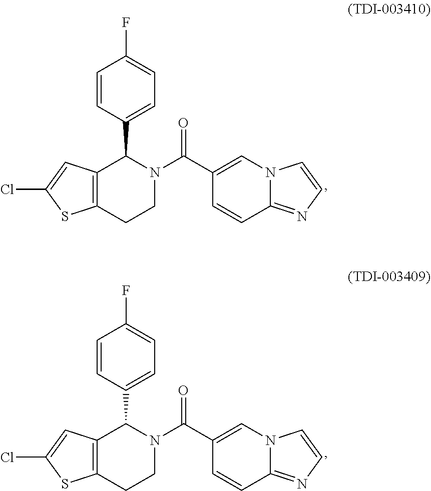 [SMILES: O=C(c1ccc2nccn2c1)N1CCc2sc(Cl)cc2[C@H]1c1ccc(F)cc1 ; O=C(c1ccc2nccn2c1)N1CCc2sc(Cl)cc2[C@@H]1c1ccc(F)cc1]

and salts, hydrates, solvates, polymorphs, co-crystals, tautomers, stereoisomers, isotopically labeled derivatives, and prodrugs thereof.

In certain embodiments, a compound of Formula (I) or Formula (II) is of one of the following formulae:

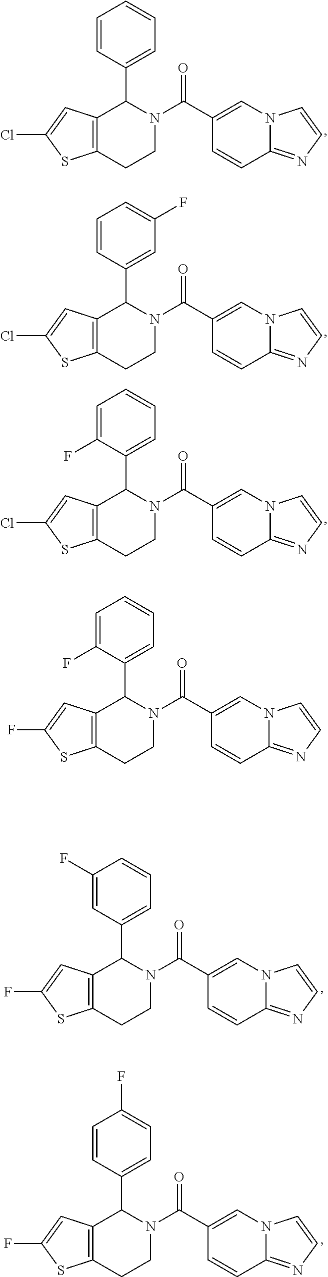

or a pharmaceutically acceptable salt, hydrate, solvate, polymorph, co-crystal, tautomer, stereoisomer, isotopically labeled derivative, or prodrug thereof.

In another aspect, the present invention provides compounds of Formula (III):

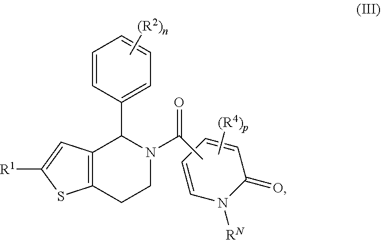

and salts, hydrates, solvates, polymorphs, co-crystals, tautomers, stereoisomers, isotopically labeled derivatives, and prodrugs thereof, wherein R¹, R², R³, R⁴, R^{N}, n, and p are as defined herein.

For example, in certain embodiments, a compound of Formula (III) is of the following formula:

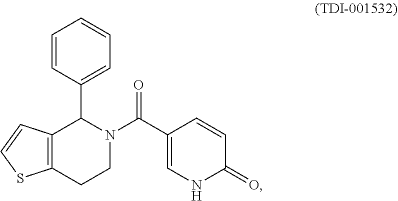 [SMILES: O=C(c1ccc(=O)[nH]c1)N1CCc2sccc2C1c1ccccc1]

or a pharmaceutically acceptable salt, hydrate, solvate, polymorph, co-crystal, tautomer, stereoisomer, isotopically labeled derivative, or prodrug thereof.

Exemplary compounds of Formula (III) include, but are not limited to, the following:

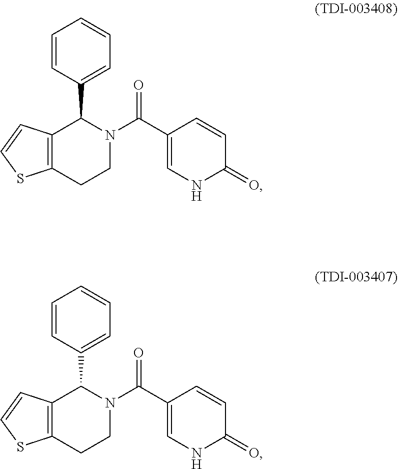 [SMILES: O=C(c1ccc(=O)[nH]c1)N1CCc2sccc2[C@H]1c1ccccc1 ; O=C(c1ccc(=O)[nH]c1)N1CCc2sccc2[C@@H]1c1ccccc1]

and salts, hydrates, solvates, polymorphs, co-crystals, tautomers, stereoisomers, isotopically labeled derivatives, and prodrugs thereof.

In certain embodiments, a compound of Formula (III) is of the following formula:

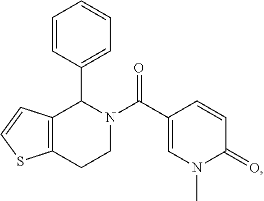 [SMILES: Cn1cc(C(=O)N2CCc3sccc3C2c2ccccc2)ccc1=O]

or a pharmaceutically acceptable salt, hydrate, solvate, polymorph, co-crystal, tautomer, stereoisomer, isotopically labeled derivative, or prodrug thereof.

In another aspect, the present invention provides pharmaceutical compositions comprising a compound of Formula (I), (II), or (III), or a pharmaceutically acceptable salt, hydrate, solvate, polymorph, co-crystal, tautomer, stereoisomer, isotopically labeled derivative, or prodrug thereof, and optionally a pharmaceutically acceptable excipient. In certain embodiments, the pharmaceutical compositions described herein include a therapeutically effective amount of a compound of Formula (I), (II), or (III), or a pharmaceutically acceptable salt, solvate, hydrate, polymorph, co-crystal, tautomer, stereoisomer, isotopically labeled derivative, or prodrug thereof. The pharmaceutical compositions described herein may be useful for treating and/or preventing a disease or condition (e.g., proliferative diseases, such as cancers and inflammatory diseases) in a subject.

In another aspect, the present invention provides methods for treating and/or preventing a disease in a subject. The method may comprise administering to a subject in need thereof a therapeutically effective amount of a compound of Formula (I), (II), or (III), or a pharmaceutically acceptable salt, hydrate, solvate, polymorph, co-crystal, tautomer, stereoisomer, isotopically labeled derivative, or prodrug thereof, or a pharmaceutical composition thereof. In certain embodiments, the disease is a proliferative disease, such as cancer. In particular embodiments, the disease is a cancer (e.g., pancreatic cancer, breast cancer, lung cancer (e.g., squamous cell carcinoma)). In certain embodiments, the disease is an inflammatory disease (e.g., arthritis). In certain embodiments, the disease is an autoimmune disease.

Compound provided herein are effective Hhat inhibitors. Therefore, the present invention also provides methods for inhibiting Hedgehog acyltransferase (Hhat) using the compounds described herein. A compound of Formula (I), (II), or (III), or a salt, hydrate, solvate, polymorph, co-crystal, tautomer, stereoisomer, isotopically labeled derivative, or prodrug thereof, or a pharmaceutical composition thereof, can be used to inhibit an Hhat. The method of inhibiting an Hhat can occur in vivo (e.g., in a subject) or in vitro (e.g., an assay). In certain embodiments, the method of inhibiting Hhat comprises contacting the Hhat with a compound of Formula (I), (II), or (III), or a salt, hydrate, solvate, polymorph, co-crystal, tautomer, stereoisomer, isotopically labeled derivative, or prodrug thereof, or a pharmaceutical composition thereof.

In yet another aspect, the present invention provides a method for inducing apoptosis using a compound of Formula (I), (II), or (III), or a salt, hydrate, solvate, polymorph, co-crystal, tautomer, stereoisomer, isotopically labeled derivative, or prodrug thereof, or a pharmaceutical composition thereof. The inventive compounds can be used to induce apoptosis in vivo or in vitro. In certain embodiments, the method of inducing apoptosis comprises contacting a cell with a compound of Formula (I), (II), or (III), or a salt, hydrate, solvate, polymorph, co-crystal, tautomer, stereoisomer, isotopically labeled derivative, or prodrug thereof, or a pharmaceutical composition thereof. In certain embodiments, the cell is a cancer cell (e.g., pancreatic cancer cell, breast cancer cell, lung cancer cell).

The present invention also provides uses of compounds of Formulae (I), (II), or (III), or pharmaceutically acceptable salts, hydrates, solvates, polymorphs, co-crystals, tautomers, stereoisomers, isotopically labeled derivatives, or prodrugs thereof, or pharmaceutical compositions thereof, for the treatment of diseases and/or conditions, for inhibiting Hhat, for inducing apoptosis, etc.

Another aspect of the present invention relates to kits comprising a compound of Formula (I), (II), or (III), or a salt, hydrate, solvate, polymorph, co-crystal, tautomer, stereoisomer, isotopically labeled derivative, or prodrug thereof, or pharmaceutical composition thereof. The kits described herein may include a single dose or multiple doses of the compound or pharmaceutical composition thereof. The provided kits may be useful in a method of the invention (e.g., a method of treating and/or preventing a disease in a subject). A kit of the invention may further include instructions for using the kit (e.g., instructions for using the compound or pharmaceutical composition included in the kit).

The details of certain embodiments of the invention are set forth herein. Other features, objects, and advantages of the invention will be apparent from the Detailed Description, Figures, Examples, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

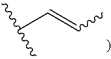

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 t electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl, and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

The term “unsaturated bond” refers to a double or triple bond. The term “unsaturated” or “partially unsaturated” refers to a moiety that includes at least one double or triple bond. The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂R^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{a}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl) ⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein. The term “substituted with oxygen” refers to a group that is substituted with hydroxyl or substituted hydroxyl.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “substituted with nitrogen” refers to a group that is substituted with amino or substituted amino.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{a}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate. Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, o-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, F⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

### Other Definitions

The following definitions are more general terms used throughout the present application.

“Hedgehog acyltransferase” (abbreviated herein as “Hhat”) refers to a member of the membrane-bound O-acyl transferase (MBOAT) family of proteins. Hhat catalyzes the post-translational modification of proteins including, but not limited to, Sonic hedgehog (abbreviated herein as “Shh”). In particular, Hhat is one of three MBOAT proteins responsible for ligating fatty acids to proteins, and is responsible for the palmitoylation of Shh. Palmitoylation of Shh is critical to the Shh signaling pathway, and therefore the activity of Hhat is crucial to proper Shh signaling.

“Sonic hedgehog” refers to the protein that in humans is encoded by the Sonic hedgehog gene. In the mammalian Hedgehog family signaling pathway, Sonic hedgehog is one of three proteins. Other proteins in the Hedgehog signaling pathway in mammals are Desert hedgehog (Dhh) and Indian hedgehog (Ihh). In developing mammals, Shh plays key roles, including regulation of limb development and organization of the brain. In adult mammals, Shh controls cell division of adult stem cells. Shh expression is turned off in most post-embryonic cells, and aberrant Shh signaling has been linked to the development of various diseases (e.g., proliferative diseases, such as cancers and inflammatory diseases).

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The term “co-crystal” refers to a crystalline structure comprising at least two different components (e.g., a compound of Formula (I), (II), or (III) and an acid), wherein each of the components is independently an atom, ion, or molecule. In certain embodiments, none of the components is a solvent. In certain embodiments, at least one of the components is a solvent. A co-crystal of a compound of Formula (I), (II), or (III) and an acid is different from a salt formed from a compound of Formula (I), (II), or (III) and the acid. In the salt, the compound is complexed with the acid in a way that proton transfer (e.g., a complete proton transfer) from the acid to the compound easily occurs at room temperature. In the co-crystal, however, the compound is complexed with the acid in a way that proton transfer from the acid to the compound does not easily occur at room temperature. In certain embodiments, in the co-crystal, there is no proton transfer from the acid to the compound. In certain embodiments, in the co-crystal, there is partial proton transfer from the acid to the compound. Co-crystals may be useful to improve the properties (e.g., solubility, stability, and ease of formulation) of a compound of Formula (I), (II), or (III).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a proliferative disease. In certain embodiments, a therapeutically effective amount is an amount sufficient for treating cancer. In certain embodiments, a therapeutically effective amount is an amount sufficient for treating leukemia. In certain embodiments, a therapeutically effective amount is an amount sufficient for treating acute myeloid leukemia.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., acute monocytic leukemia, acute myelomonocytic leukemia, acute promyelocytic leukemia), chronic myelocytic leukemia (CML), and chronic lymphocytic leukemia (CLL)); myelodysplastic syndromes; myeloproliferative neoplasms; myelofibrosis; lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrim's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphomalleukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemiallymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

“Antiproliferative agents” include “anti-cancer agents”, and encompass biotherapeutic anti-cancer agents as well as chemotherapeutic agents. Exemplary biotherapeutic anti-proliferative agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune checkpoint inhibitors, chemokine receptor inhibitors, immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents (i.e., chemotherapeutic anti-proliferative agents) include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe), and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

As used herein, the term “apoptosis” refers to a regulated network of biochemical events which lead to a selective form of cell suicide and is characterized by readily observable morphological and biochemical phenomena. Cells undergoing apoptosis show characteristic morphological and biochemical features. These features include chromatin aggregation or condensation, DNA fragmentation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial outer membrane permeabilization accompanying apoptosis.

As used herein, “inhibition”, “inhibiting”, “inhibit” and “inhibitor”, and the like, refer to the ability of a compound to reduce, slow, halt, or prevent the activity of a biological process (e.g., a biological process in a cell). In certain embodiments, such inhibition is of about 1% to 99.9%. In certain embodiments, the inhibition is about 1% to about 95%. In certain embodiments, the inhibition is about 5% to 90%. In certain embodiments, the inhibition is about 10% to 85%. In certain embodiments, the inhibition is about 15% to 80%. In certain embodiments, the inhibition is about 20% to 75%. In certain embodiments, the inhibition is about 25% to 70%. In certain embodiments, the inhibition is about 30% to 65%. In certain embodiments, the inhibition is about 35% to 60%. In certain embodiments, the inhibition is about 40% to 55%. In certain embodiments, the inhibition is about 45% to 50%. In certain embodiments, the inhibition is about 5%, 10%, 15%, 20%, 25%, 30%, 35%, 40%, 45%, 50%, 55%, 60%, 65%, 70%, 75%, 80%, 85%, 90%, 95%, or 99.9%.

### BRIEF DESCRIPTION OF THE DRAWINGS

The accompanying drawings, which constitute a part of this specification, illustrate several embodiments of the invention and together with the description, serve to explain the principles of the invention.

FIGS. 1A-1B. TDI-003410 blocks Hhat-mediated Shh palmitoylation. FIG. 1A. COS-1 cells were transfected with cDNA plasmids encoding Shh and Hhat (see, e.g., Buglino, J. A., and Resh, M. D. “Hhat is a palmitoyl acyltransferase with specificity for N-palmitoylation of sonic hedgehog” J. Biol. Chem. 2008, 283, 22076-22088; Petrova, E., Rios-Esteves, J., Ouerfelli, O., Glickman, J. F., and Resh, M. D. “Inhibitors of Hedgehog acyltransferase block Sonic Hedgehog signaling” Nat Chem Biol 2013, 9, 247-249). 48 hours after transfection, cells were starved for 1 hour in DMEM containing 2% dialyzed fetal calf serum and then labeled for 4 hr at 37° C. with 15-30 mCi/ml [¹²⁵I]-iodopalmitate in the presence of 10 mM of the indicated compound or DMSO. Cell lysates were immunoprecipitated with anti-Shh antibody, analyzed by SDS-PAGE and dried gels were subjected to Phosphorimaging. The amount of radiolabel incorporated into Shh was quantified and compared to that obtained for DMSO (normalized to 1.0). % Inhibition=1−(signal in drug treated sample/signal in DMSO sample)×100. FIG. 1B. Structure of RU-SKI 43 (see, e.g., Petrova, E., Rios-Esteves, J., Ouerfelli, O., Glickman, J. F., and Resh, M. D. “Inhibitors of Hedgehog acyltransferase block Sonic Hedgehog signaling” Nat Chem Biol 2013, 9, 247-249).

FIGS. 2A-2D show inhibition of the growth of AsPC1 cells by TDI compounds. AsPC1 human pancreatic cancer cells were seeded in 24-well plates (0.5×10⁵ cells/well) and grown for 6 days in the presence of 10 mM of the indicated compound or DMSO. Compounds were added on Day 0, and were replenished in the media every 2 days. Each condition was represented by duplicate wells. Cell number was quantified by Crystal violet staining. FIG. 2A. Growth curve. FIG. 2B. Crystal violet staining of a typical set of 6 wells. FIG. 2C. Bar graph depicting % inhibition of cell proliferation at Day 6 for each compound, compared to DMSO treated cells. % Inhibition was calculated as in FIG. 1A. FIG. 2D is an inhibition curve for TDI-003410; IC₅₀=2.68 M.

FIG. 3. TDI-003410 inhibits Shh palmitoylation by purified Hhat. Pulldown assay: Purified Hhat+¹²⁵I-palmitoyl CoA+Shh peptide. IC₅₀=1.5 μM. FIG. 3 shows TDI-3410 inhibits Shh palmitoylation by purified Hhat. Hhat was purified to homogeneity from 293FT cells transfected with a cDNA encoding HA-tagged Hhat, as described by Buglino and Resh (see, e.g., Buglino, J. A., and Resh, M. D. “Hhat is a palmitoyl acyltransferase with specificity for N-palmitoylation of sonic hedgehog” J. Biol. Chem. 2008, 283, 22076-22088). Purified Hhat was incubated with ¹²⁵I-Iodopalmitoyl CoA and a C-terminally biotinylated Shh peptide (representing the first 10 amino acids of mature Shh) for 1 hr at 20° C. in the presence of the indicated concentrations of TDI-3410. The reaction was quenched, and biotinylated peptides were captured on streptavidin-agarose beads by repeated centrifugation and washing. The amount of 125 radioactivity remaining in the bead pellet was determined in a g counter. % Inhibition was calculated as in FIG. 1A.

FIG. 4 shows TDI-003410 inhibits Shh palmitoylation by membrane-bound Hhat. Microsomal membranes (P100) were prepared from 293FT cells transfected with a cDNA encoding HA-tagged Hhat as described (see, e.g., Buglino, J. A., and Resh, M. D. “Hhat is a palmitoyl acyltransferase with specificity for N-palmitoylation of sonic hedgehog” J. Biol. Chem. 2008, 283, 22076-22088; Petrova, E., Rios-Esteves, J., Ouerfelli, O., Glickman, J. F., and Resh, M. D. “Inhibitors of Hedgehog acyltransferase block Sonic Hedgehog signaling” Nat Chem Biol 2013, 9, 247-249). The protocol described by Petrova et al. (Nat Chem Biol 2013, 9, 247-249) was used to analyze Shh palmitoylation. Briefly, P100 membranes were incubated with ¹²⁵I-Iodopalmitoyl CoA and a C-terminally biotinylated Shh peptide in 384-well plates in the presence of the indicated concentrations of TDI-3410 for 1 hr at 20° C. Biotinylated peptides were captured on streptavidin-coated SPA (Scintillation Proximity Assay) beads and the amount of ¹²⁵I radioactivity was quantified on a Perkin-Elmer Trilux reader. Each data point represents the average of triplicate samples. % Inhibition was calculated as in FIG. 1A. The calculated IC₅₀=1.78 μM.

FIGS. 5A-5B show TDI-003410 inhibits the growth of human pancreatic and breast cancer cells. AsPC1 human pancreatic cancer cells (FIG. 5A) or T47D human breast cancer cells (FIG. 5B) were seeded at 750 cells/well in 96 well plates and grown for 6 days in the presence of the indicated concentrations of TDI-3410 or DMSO. Compounds were added on Day 0, and were replenished in the media every 2 days. Each condition was represented by duplicate wells. Cell number was quantified at Day 6 using EZQuant (Alstem).

FIGS. 6A-B show that HeLa cells do not express detectable levels of Hhat and are not sensitive to Hhat inhibitors. 24-well plate; Drugs replenished every 2 days; viable cells quantified with EZQuant; STS=Staurosporine, a positive control for cell killing. FIG. 6A shows TDI-003410 has little to no effect on proliferation of HeLa cells. HeLa cells were seeded at 0.5×10⁵ cells/well in 24-well plates and proliferation was assayed as described for FIG. 5. Each compound was present at 10 mM. Compound 2 is a control compound that does not inhibit Hhat (see, e.g., Petrova, E., Rios-Esteves, J., Ouerfelli, O., Glickman, J. F., and Resh, M. D. “Inhibitors of Hedgehog acyltransferase block Sonic Hedgehog signaling” Nat Chem Biol 2013, 9, 247-249). STS=Staurosporine, a positive control for cell killing. qRT-PCR analyses indicate that HeLa cells do not express detectable levels of Hhat mRNA. FIG. 6B is an inhibition curve for TDI-003410, which shows that TDI-003410 does not inhibit the growth of HeLa cells until the concentration of TDI-003410 is greater 20 μM; IC₅₀=28.47 μM.

FIGS. 7A-7B show TDI-3410 (TDI-003410) inhibits the growth of H1703 oncospheres. H1703 cells, a human lung squamous cell carcinoma cell line, were grown in serum-free media containing growth factors in 24-well low-adherence tissue culture plates to generate oncospheres, cell spheres that exhibit properties of cancer stem cells (see, e.g., Justilien, V., Walsh, M. P., Ali, S. A., Thompson, E. A., Murray, N. R., and Fields, A. P. “The PRKCI and SOX2 oncogenes are coamplified and cooperate to activate Hedgehog signaling in lung squamous cell carcinoma” Cancer Cell 2014, 25, 139-151). Duplicate wells were treated with the indicated concentrations of compound for 10 days. Compounds were replenished in the media every 2 days. Cell number was quantified at Day 10 using EZQuant (Alstem). FIG. 7A shows curves demonstrating reduction in cell count with increased concentration of TDI-3410, TDI-3409, or RU-SKI 43. FIG. 7B shows the inhibition curve for TDI-003410; IC₅₀=1.4 μM.

FIG. 8 is an inhibition curve, showing that TDI-3410 also inhibits the growth of oncospheres derived from AsPC-1 human pancreatic cancer cells. AsPC-1 pancreatic cancer cells were grown on ultra-low attachment plates for 7-10 days in serum-free medium containing EGF, FGF, Insulin and N-2 and B-27 supplement to form oncospheres, which represent cancer stem cells. Oncosphere cultures were expanded and replated, and were used after 7 days in 2° culture. Oncospheres grown in 24-well plates were treated with TDI-3410 or vehicle; drug was replenished every 2 days. 7 days later, oncospheres were dissociated with trypsin and total viable cell number was determined using EZQuant. The experiment in FIG. 8 revealed that TDI-3410 inhibits cell proliferation with an IC₅₀=1.8 μM.

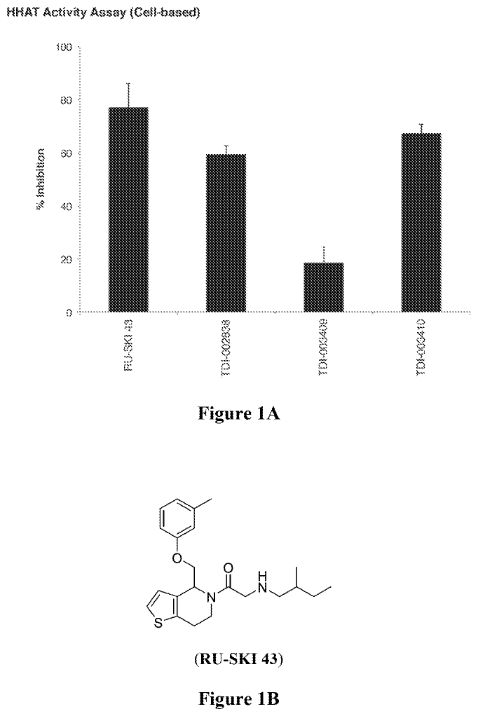

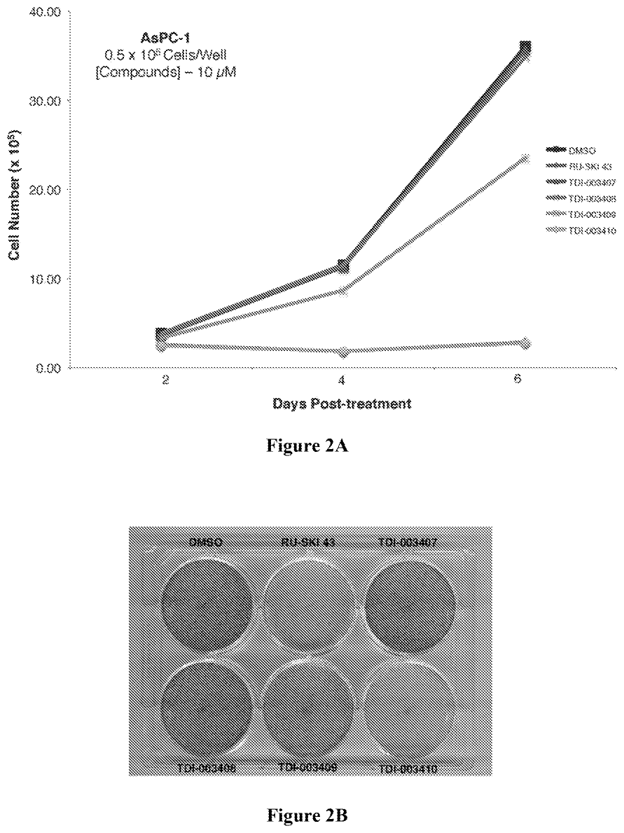

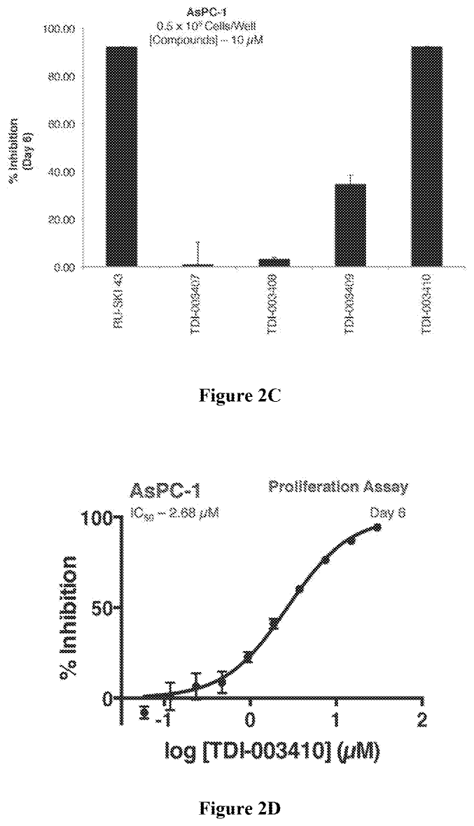

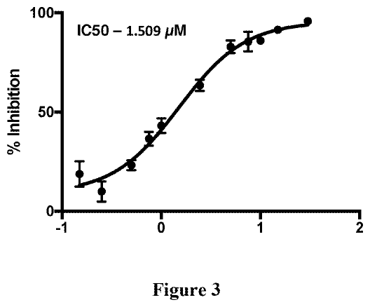

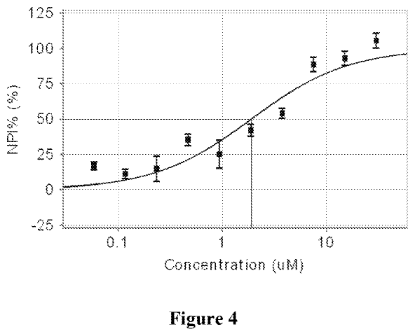

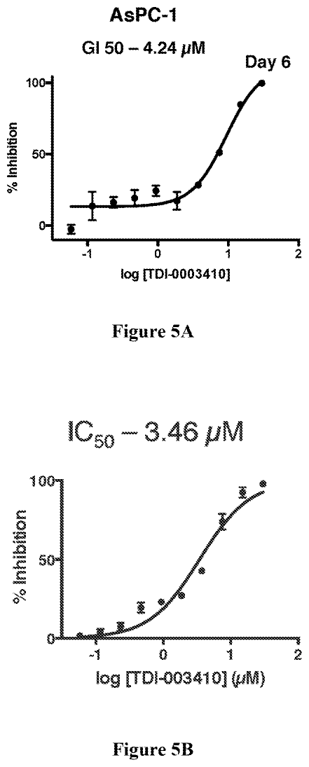

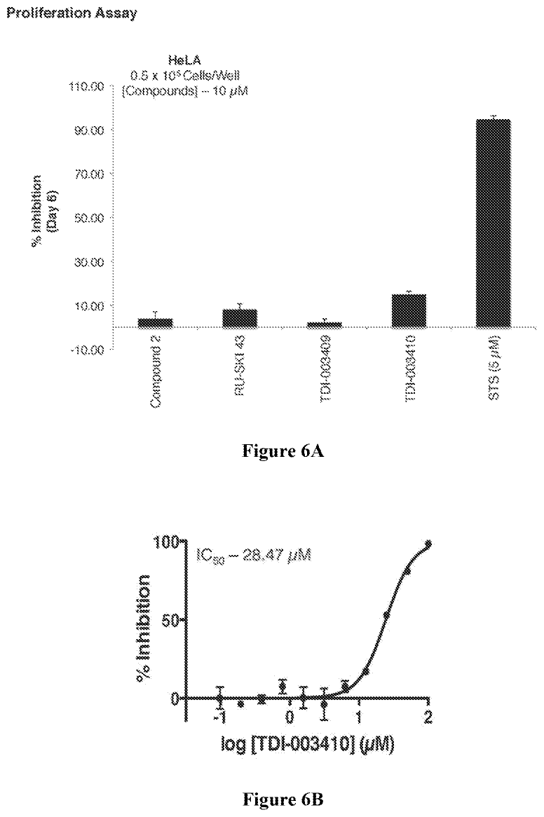

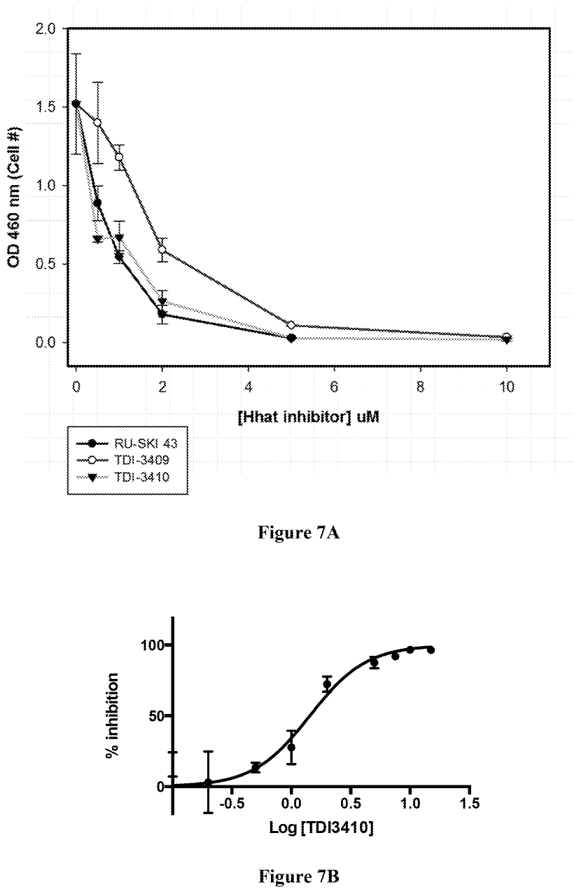

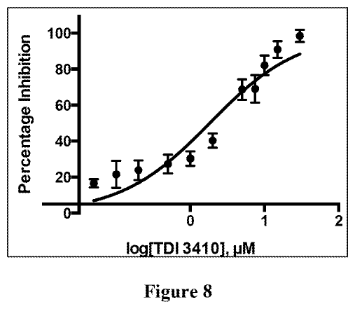

### DETAILED DESCRIPTION OF CERTAIN EMBODIMENTS

The Sonic hedgehog (Shh) signaling pathway has been linked to the development of diseases, including proliferative diseases (e.g., cancer and inflammatory diseases). Sonic hedgehog undergoes post-translational modifications that are critically important to its signaling capabilities, including the ligation of fatty acids such as palmitate. Hedgehog acyltransferase (Hhat), a membrane-bound O-acyl transferase (MBOAT) protein, is responsible for the post-translational modification of hedgehog proteins (e.g., Shh, Dhh, Ihh). Specifically, Hhat is responsible for the palmitoylation of Shh and is crucial to proper Shh signaling. Hhat inhibitors that are capable of preventing Shh palmitoylation and mitigating Shh signaling, and therefore can be used in the treatment and/or prevention of diseases (e.g., proliferative diseases, such as cancer, autoimmune diseases, and inflammatory diseases). Hedgehog acyltransferase may also be involved in non-canonical pathways (e.g., pathways not involving the Shh-Patched-Smoothened-Gli signaling axis), and therefore other signaling pathways could be affected by Hhat inhibition. Provided herein are Hhat inhibitors, such as compounds of Formulae (I), (II), and (III), which are useful for the treatment and/or prevention of diseases. Also provided herein are pharmaceutical compositions comprising a compound of Formulae (I), (II), or (III). As discussed below, the present invention also provides methods of using the inventive compounds and pharmaceutical compositions described herein (e.g., for treating and/or preventing diseases, for inhibiting Hhat, for inducing apoptosis).

### Compounds

Provided herein are compounds of Formula (I):

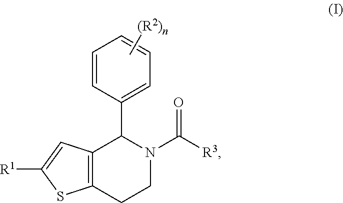

and pharmaceutically acceptable salts, hydrates, solvates, polymorphs, co-crystals, tautomers, stereoisomers, isotopically labeled derivatives, or prodrugs thereof, wherein:

R¹ is halogen or optionally substituted alkyl;

each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};

n is 1, 2, 3, 4, or 5;

R³ is optionally substituted monocyclic or bicyclic carbocyclyl, optionally substituted monocyclic or bicyclic aryl, optionally substituted monocyclic or bicyclic heterocyclyl, or optionally substituted monocyclic or bicyclic heteroaryl;

each instance of R^{2a} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group;

each instance of R^{2b} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or optionally, two R^{2b} are taken together with the intervening atoms to form optionally substituted heterocyclyl or heteroaryl; and

each instance of R^{2c} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

In certain embodiments, the compound of Formula (I) is of one of the following formulae:

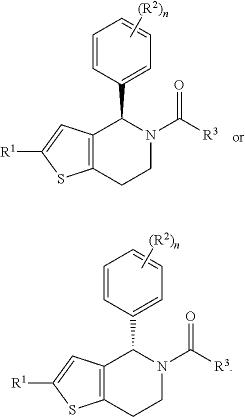

In certain embodiments, a compound of Formula (I), (II), or (III) comprises a non-hydrogen substituent at the positions corresponding to R¹. Without wishing to be bound by a particular theory, including an electron-withdrawing group at this position (e.g., halogen) may help confer metabolic stability by mitigating metabolic pathways involving the thiophene moiety (e.g., oxidation of the thiophene moiety). Therefore, certain compounds of the present invention include a non-hydrogen substituent (e.g., halogen) at R¹.

In certain embodiments, R¹ is halogen. In certain embodiments, R¹ is Cl; and the compound of Formula (I) is of Formula (I-a):

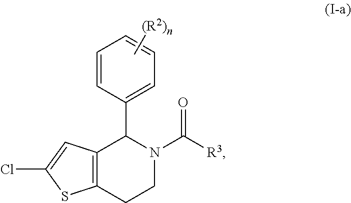

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (I-a) is of the following formula:

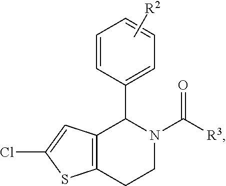

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (I-a) is of the following formula:

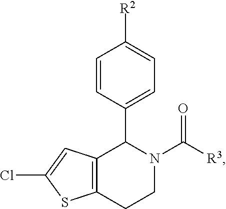

or a pharmaceutically acceptable salt, hydrate, solvate, polymorph, co-crystal, tautomer, stereoisomer, isotopically labeled derivative, or prodrug thereof.

In certain embodiments, n is 1; and R² is halogen. In certain embodiments, n is 1; R² is F; and the compound of Formula (I-a) is of the following formula:

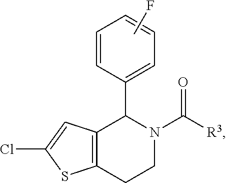

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (I) is of the following formula:

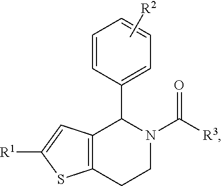

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (I) is of the following formula:

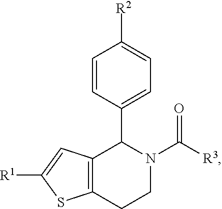

or a pharmaceutically acceptable salt, hydrate, solvate, polymorph, co-crystal, tautomer, stereoisomer, isotopically labeled derivative, or prodrug thereof.

In certain embodiments, n is 1; R² is F; and the compound of Formula (I) is of the following formula:

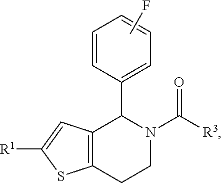

or a pharmaceutically acceptable salt, hydrate, solvate, polymorph, co-crystal, tautomer, stereoisomer, isotopically labeled derivative, or prodrug thereof.

In certain embodiments, n is 1; R² is F; and the compound of Formula (I) is of Formula (I-b):

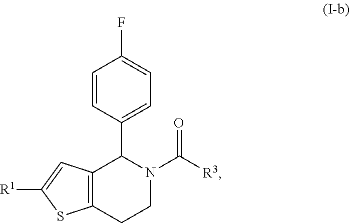

or a pharmaceutically acceptable salt, hydrate, solvate, polymorph, co-crystal, tautomer, stereoisomer, isotopically labeled derivative, or prodrug thereof.

In certain embodiments, R¹ is Cl; and the compound of Formula (I-b) is of Formula (I-c):

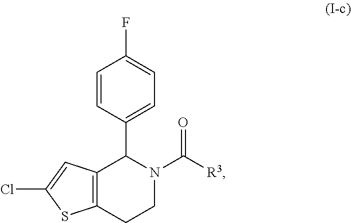

or a pharmaceutically acceptable salt, hydrate, solvate, polymorph, co-crystal, tautomer, stereoisomer, isotopically labeled derivative, or prodrug thereof.

In another aspect, provided herein are compounds of Formula (II):

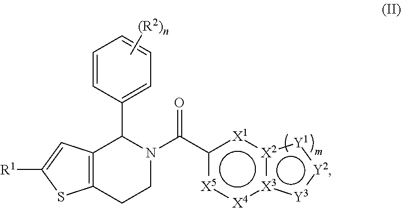

and pharmaceutically acceptable salts, hydrates, solvates, polymorphs, co-crystals, tautomers, stereoisomers, isotopically labeled derivatives, and prodrugs thereof, wherein:

R¹ is hydrogen, halogen, or optionally substituted alkyl;

each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};

n is 1, 2, 3, 4, or 5;

m is 1 or 2;

each instance of X¹, X², X³, X⁴, X⁵, Y¹, Y², and Y³ is independently C, CR^{C}, N, NR^{N}, S, or O, as valency permits; provided that at least one of X¹, X², X³, X⁴, or X⁵ is N, NR^{N}, S, or O;

each instance of R^{C} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};

each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group;

each instance of R^{2a} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or an oxygen protecting group;

each instance of R^{2b} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or a nitrogen protecting group; or optionally, two R^{2b} are taken together with the intervening atoms to form optionally substituted heterocyclyl or heteroaryl;

each instance of R^{2c} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or a sulfur protecting group.

In certain embodiments, the compound of Formula (II) is of one of the following formulae:

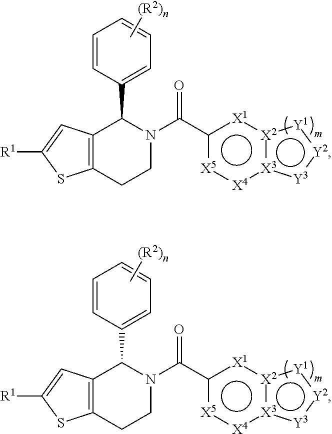

or a pharmaceutically acceptable salt, hydrate, solvate, polymorph, co-crystal, tautomer, stereoisomer, isotopically labeled derivative, or prodrug thereof.

In certain embodiments, R¹ is halogen. In certain embodiments, R¹ is Cl; and the compound of Formula (II) is of the following formula:

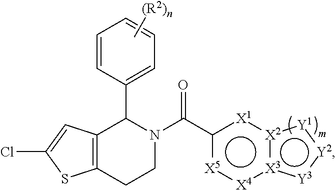

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (II) is of the following formula:

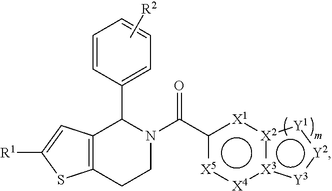

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (II) is of the following formula:

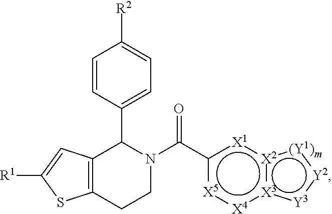

or a pharmaceutically acceptable salt, hydrate, solvate, polymorph, co-crystal, tautomer, stereoisomer, isotopically labeled derivative, or prodrug thereof.

In certain embodiments, n is 1; m is 1; and the compound of Formula (II) is of the following formula:

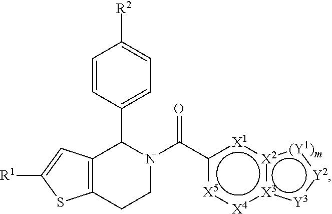

or a pharmaceutically acceptable salt, hydrate, solvate, polymorph, co-crystal, tautomer, stereoisomer, isotopically labeled derivative, or prodrug thereof.

In certain embodiments, n is 1; R² is F; and the compound of Formula (II) is of the following formula:

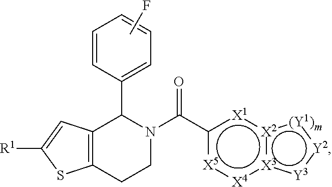

or a pharmaceutically acceptable salt, hydrate, solvate, polymorph, co-crystal, tautomer, stereoisomer, isotopically labeled derivative, or prodrug thereof.

In certain embodiments, n is 1; R² is F; and the compound of Formula (II) is of the following formula:

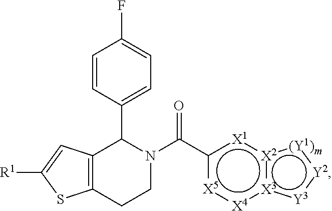

or a pharmaceutically acceptable salt, hydrate, solvate, polymorph, co-crystal, tautomer, stereoisomer, isotopically labeled derivative, or prodrug thereof.

In certain embodiments, R¹ is Cl; n is 1; R² is F; and the compound of Formula (II) is of the following formula:

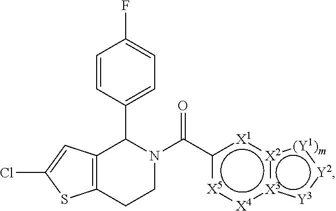

or a pharmaceutically acceptable salt, hydrate, solvate, polymorph, co-crystal, tautomer, stereoisomer, isotopically labeled derivative, or prodrug thereof.

In certain embodiments, R¹ is Cl; n is 1; R² is F; and the compound of Formula (II) is of the following formula:

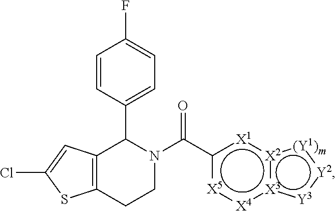

or a pharmaceutically acceptable salt, hydrate, solvate, polymorph, co-crystal, tautomer, stereoisomer, isotopically labeled derivative, or prodrug thereof.

In certain embodiments, m is 1; and the compound of Formula (II) is of the following formula:

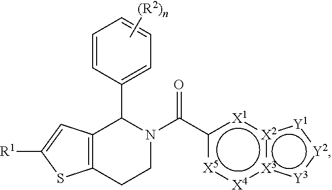

or a pharmaceutically acceptable salt, hydrate, solvate, polymorph, co-crystal, tautomer, stereoisomer, isotopically labeled derivative, or prodrug thereof.

As described herein, at least one of X¹, X², X³, X⁴, or X⁵ is N, NR^{N}, S, or O, as valency permits. In certain embodiments, at least one of X¹, X², X³, X⁴, and X⁵ is N. In certain embodiments, the compound of Formula (II) is of one of the following formulae:

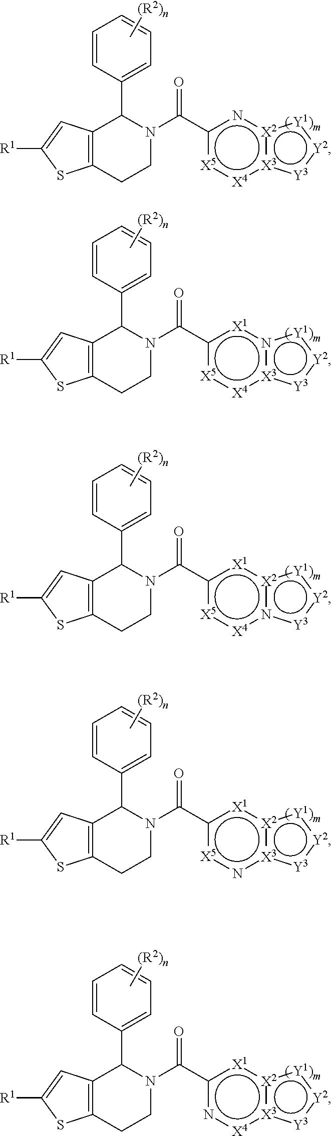

or a pharmaceutically acceptable salt, hydrate, solvate, polymorph, co-crystal, tautomer, stereoisomer, isotopically labeled derivative, or prodrug thereof.

In certain embodiments, one of X¹, X², X³, X⁴, and X⁵ is N; and the rest are CR^{C}. In certain embodiments, the compound of Formula (II) is of one of the following formulae:

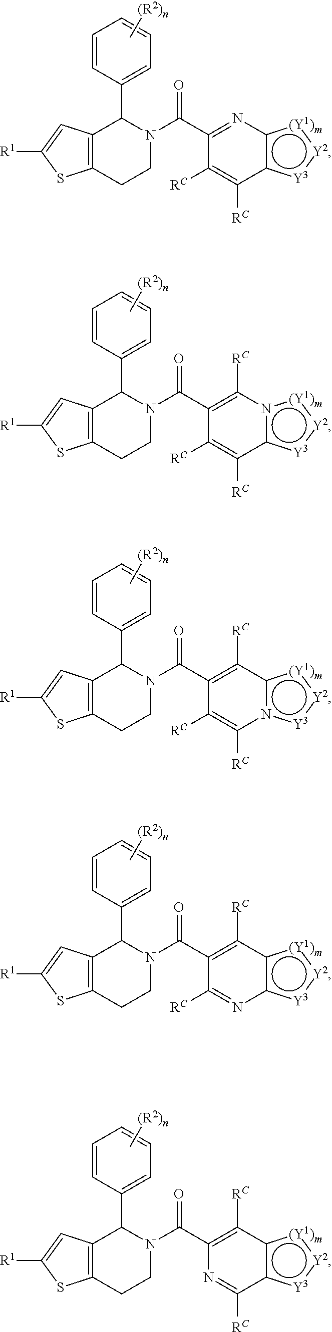

or a pharmaceutically acceptable salt, hydrate, solvate, polymorph, co-crystal, tautomer, stereoisomer, isotopically labeled derivative, or prodrug thereof.

In certain embodiments, m is 1; X² is N; X¹, X³, X⁴, and X⁵ are CR^{C}; one of Y¹, Y², and Y³ are N, and the rest are CR^{C}. For example, in certain embodiments, a compound of Formula (II) is of one of the following formulae:

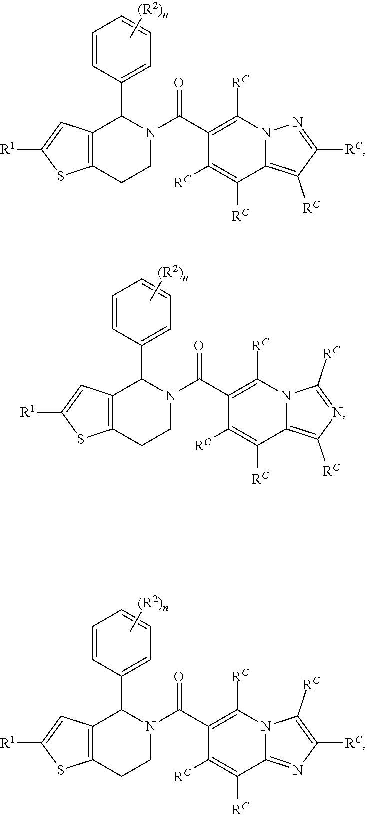

or a pharmaceutically acceptable salt, hydrate, solvate, polymorph, co-crystal, tautomer, stereoisomer, isotopically labeled derivative, or prodrug thereof.

In certain embodiments, a compound of Formula (II) is of Formula (II-a):

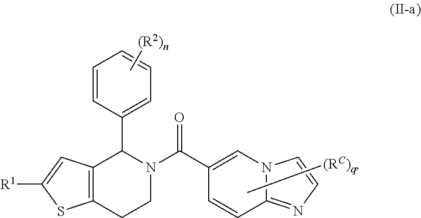

or a pharmaceutically acceptable salt, hydrate, solvate, polymorph, co-crystal, tautomer, stereoisomer, isotopically labeled derivative, or prodrug thereof, wherein:

q is 0, 1, 2, 3, 4, or 5.

In certain embodiments, R¹ is halogen. In certain embodiments, R¹ is Cl; and the compound of Formula (II-a) is of Formula (II-b):

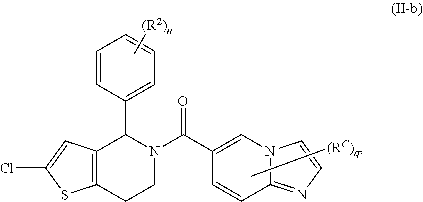

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (II-a) is of the following formula:

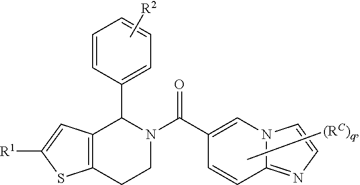

or a pharmaceutically acceptable salt, hydrate, solvate, polymorph, co-crystal, tautomer, stereoisomer, isotopically labeled derivative, or prodrug thereof.

In certain embodiments, n is 1; and the compound of Formula (II-a) is of the following formula:

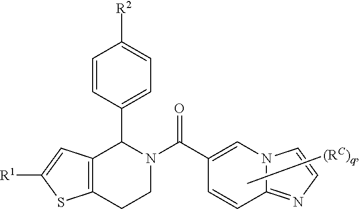

or a pharmaceutically acceptable salt, hydrate, solvate, polymorph, co-crystal, tautomer, stereoisomer, isotopically labeled derivative, or prodrug thereof.

In certain embodiments, n is 1; R² is F; and the compound of Formula (II-a) is of the following formula:

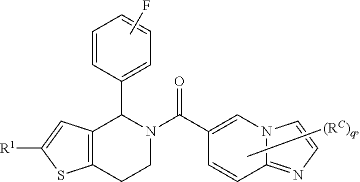

or a pharmaceutically acceptable salt, hydrate, solvate, polymorph, co-crystal, tautomer, stereoisomer, isotopically labeled derivative, or prodrug thereof.

In certain embodiments, n is 1; R² is F; and the compound of Formula (II-a) is of the following formula:

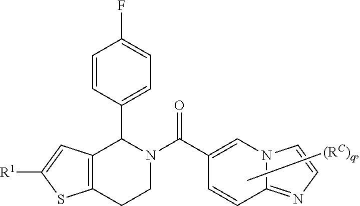

or a pharmaceutically acceptable salt, hydrate, solvate, polymorph, co-crystal, tautomer, stereoisomer, isotopically labeled derivative, or prodrug thereof.

In certain embodiments, R¹ is Cl; n is 1; R² is F; and the compound of Formula (II) is of Formula (II-c):

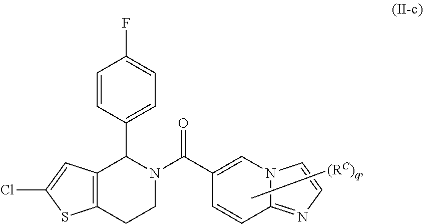

or a pharmaceutically acceptable salt, hydrate, solvate, polymorph, co-crystal, tautomer, stereoisomer, isotopically labeled derivative, or prodrug thereof.

For example, in certain embodiments, a compound of Formula (I) or Formula (II) is of the following formula:

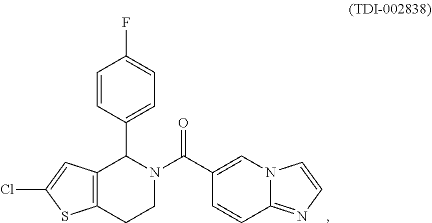 [SMILES: O=C(c1ccc2nccn2c1)N1CCc2sc(Cl)cc2C1c1ccc(F)cc1 ; ","]

or a pharmaceutically acceptable salt, hydrate, solvate, polymorph, co-crystal, tautomer, stereoisomer, isotopically labeled derivative, or prodrug thereof.

Exemplary compounds of Formula (I) and Formula (II) include, but are not limited to, the following:

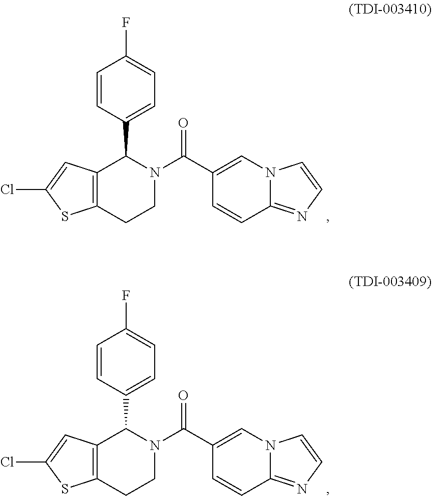 [SMILES: O=C(c1ccc2nccn2c1)N1CCc2sc(Cl)cc2[C@H]1c1ccc(F)cc1 ; "," ; O=C(c1ccc2nccn2c1)N1CCc2sc(Cl)cc2[C@@H]1c1ccc(F)cc1 ; ","]

and pharmaceutically acceptable salts, hydrates, solvates, polymorphs, co-crystals, tautomers, stereoisomers, isotopically labeled derivatives, and prodrugs thereof.

In certain embodiments, a compound of Formula (I) or Formula (II) is of one of the following formulae:

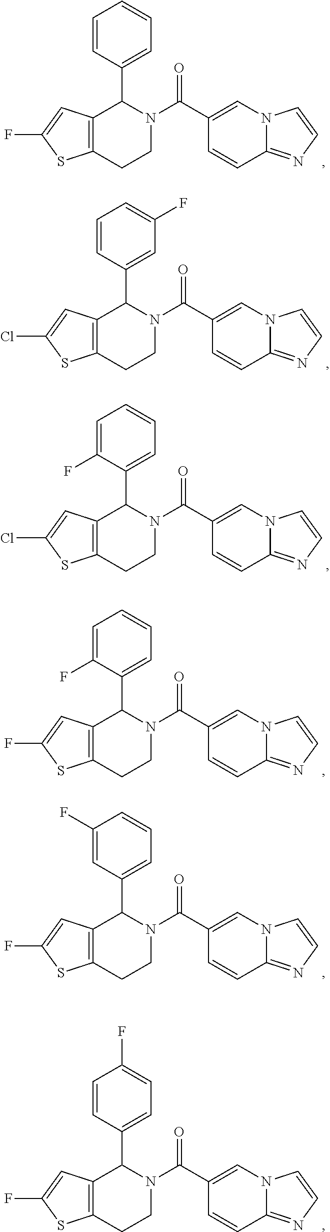

or a pharmaceutically acceptable salt, hydrate, solvate, polymorph, co-crystal, tautomer, stereoisomer, isotopically labeled derivative, or prodrug thereof.

Also provided herein are compounds of Formula (III):

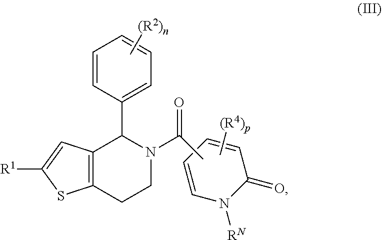

and pharmaceutically acceptable salts, hydrates, solvates, polymorphs, co-crystals, tautomers, stereoisomers, isotopically labeled derivatives, and prodrugs thereof, wherein:

R¹ is hydrogen, halogen, or optionally substituted alkyl;

each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};

each instance of R⁴ is independently hydrogen, —CN, —NO₂, —N₃, optionally substituted alkyl, optionally substituted carbocyclyl, optionally substituted aryl, optionally substituted heterocyclyl, optionally substituted five-membered heteroaryl, optionally substituted acyl, —OR^{2a}, —N(R^{2b})₂, or —SR^{2c};

each instance of R^{2a} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or an oxygen protecting group;

each instance of R^{2b} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or a nitrogen protecting group; or optionally, two R^{2b} are taken together with the intervening atoms to form optionally substituted heterocyclyl or heteroaryl;

each instance of R^{2c} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or a sulfur protecting group;

n is 1, 2, 3, 4, or 5;

p is 1, 2, or 3; and

R^{N} is hydrogen, optionally substituted alkyl, optionally substituted acyl, or a nitrogen protecting group.

In certain embodiments, the compound of Formula (III) is of one of the following formulae:

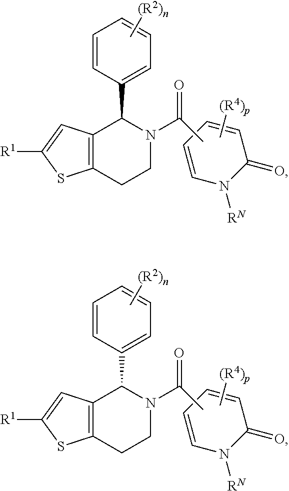

or a pharmaceutically acceptable salt, hydrate, solvate, polymorph, co-crystal, tautomer, stereoisomer, isotopically labeled derivative, or prodrug thereof.

In certain embodiments, the compound of Formula (III) is of the following formula:

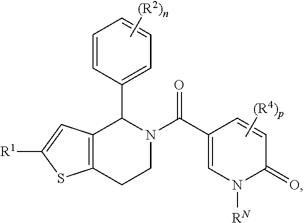

or a pharmaceutically acceptable salt, hydrate, solvate, polymorph, co-crystal, tautomer, stereoisomer, isotopically labeled derivative, or prodrug thereof.

In certain embodiments, p is 0; and the compound of Formula (III) is of the following formula:

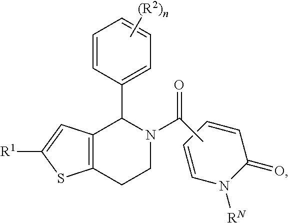

or a pharmaceutically acceptable salt, hydrate, solvate, polymorph, co-crystal, tautomer, stereoisomer, isotopically labeled derivative, or prodrug thereof.

In certain embodiments, p is 0; and the compound of Formula (III) is of the following formula:

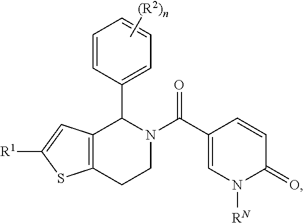

or a pharmaceutically acceptable salt, hydrate, solvate, polymorph, co-crystal, tautomer, stereoisomer, isotopically labeled derivative, or prodrug thereof.

In certain embodiments, p is 0; R^{N} is hydrogen; and the compound of Formula (III) is of the following formula:

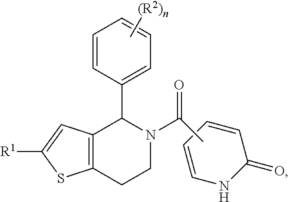

or a pharmaceutically acceptable salt, hydrate, solvate, polymorph, co-crystal, tautomer, stereoisomer, isotopically labeled derivative, or prodrug thereof.

In certain embodiments, p is 0; R^{N} is hydrogen; and the compound of Formula (III) is of the following formula:

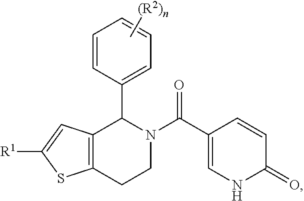

or a pharmaceutically acceptable salt, hydrate, solvate, polymorph, co-crystal, tautomer, stereoisomer, isotopically labeled derivative, or prodrug thereof.

In certain embodiments, n is 0; p is 0; and the compound of Formula (III) is of the following formula:

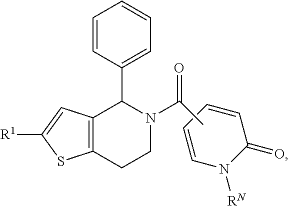

or a pharmaceutically acceptable salt, hydrate, solvate, polymorph, co-crystal, tautomer, stereoisomer, isotopically labeled derivative, or prodrug thereof.

In certain embodiments, n is 0; p is 0; R^{N} is hydrogen; and the compound of Formula (III) is of the following formula:

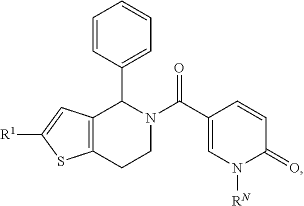

or a pharmaceutically acceptable salt, hydrate, solvate, polymorph, co-crystal, tautomer, stereoisomer, isotopically labeled derivative, or prodrug thereof.

In certain embodiments, n is 0; p is 0; R^{N} is hydrogen; and the compound of Formula (III) is of the following formula:

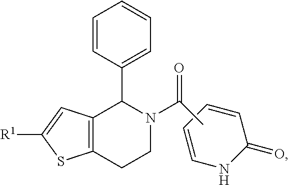

or a pharmaceutically acceptable salt, hydrate, solvate, polymorph, co-crystal, tautomer, stereoisomer, isotopically labeled derivative, or prodrug thereof.

In certain embodiments, n is 0; p is 0; R^{N} is hydrogen; and the compound of Formula (III) is of the following formula:

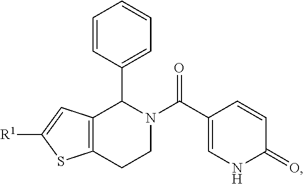

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

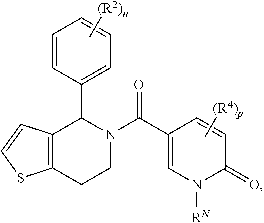

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

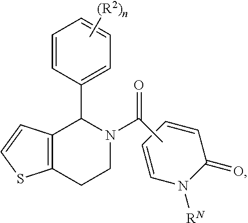

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

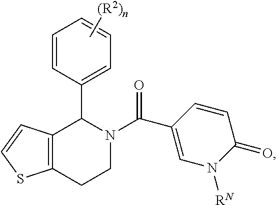

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

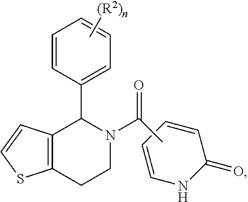

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

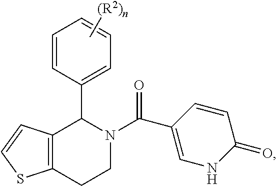

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

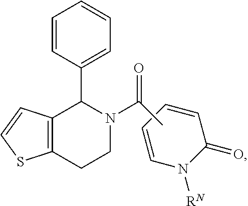

or a pharmaceutically acceptable salt, hydrate, solvate, polymorph, co-crystal, tautomer, stereoisomer, isotopically labeled derivative, or prodrug thereof.

In certain embodiments, R¹ is hydrogen; and the compound of Formula (III) is of the following formula:

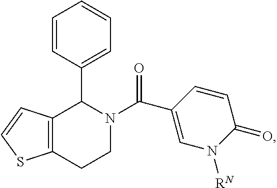

or a pharmaceutically acceptable salt, hydrate, solvate, polymorph, co-crystal, tautomer, stereoisomer, isotopically labeled derivative, or prodrug thereof.

In certain embodiments, R¹ is hydrogen; n is 0; p is 0; R^{N} is hydrogen; and the compound of Formula (III) is of Formula (III-a):

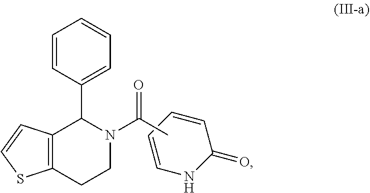

or a pharmaceutically acceptable salt, hydrate, solvate, polymorph, co-crystal, tautomer, stereoisomer, isotopically labeled derivative, or prodrug thereof.

In certain embodiments, the compound of Formula (III-a) is of the following formula:

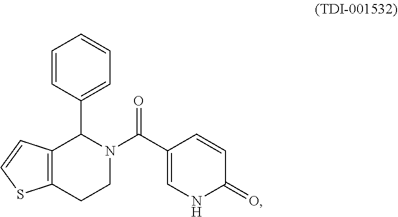 [SMILES: O=C(c1ccc(=O)[nH]c1)N1CCc2sccc2C1c1ccccc1]

or a pharmaceutically acceptable salt, hydrate, solvate, polymorph, co-crystal, tautomer, stereoisomer, isotopically labeled derivative, or prodrug thereof.

Exemplary compounds of Formula (III) include, but are not limited to, the following:

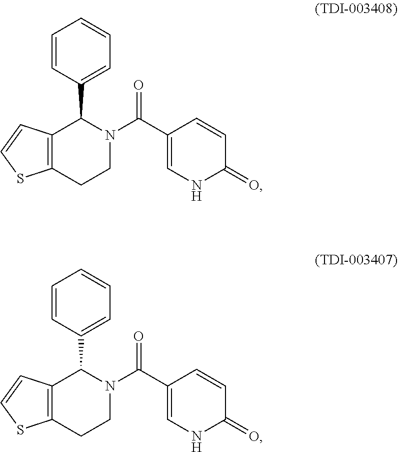 [SMILES: O=C(c1ccc(=O)[nH]c1)N1CCc2sccc2[C@H]1c1ccccc1 ; O=C(c1ccc(=O)[nH]c1)N1CCc2sccc2[C@@H]1c1ccccc1]

and pharmaceutically acceptable salts, hydrates, solvates, polymorphs, co-crystals, tautomers, stereoisomers, isotopically labeled derivatives, and prodrugs thereof.

In certain embodiments, a compound of Formula (III) is of the following formula:

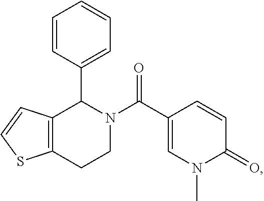 [SMILES: Cn1cc(C(=O)N2CCc3sccc3C2c2ccccc2)ccc1=O]

or a pharmaceutically acceptable salt, hydrate, solvate, polymorph, co-crystal, tautomer, stereoisomer, isotopically labeled derivative, or prodrug thereof.

The following R group definitions apply to any formulae recited herein (e.g., Formula (I), (II), or (III), and the various subgenera of Formula (I), (II), or (III)).

### Group R¹

As generally defined herein for Formula (I), R¹ may be halogen or optionally substituted alkyl. As generally defined herein for Formulae (II) and (II), R¹ may be hydrogen, halogen, or optionally substituted alkyl. In certain embodiments, R¹ is hydrogen. In certain embodiments, R¹ is optionally substituted alkyl. In certain embodiments, R¹ is optionally substituted C₁₋₆ alkyl. In certain embodiments, R¹ is substituted C₁₋₆ alkyl. In certain embodiments, R¹ is unsubstituted C₁₋₆ alkyl. In certain embodiments, R¹ is optionally substituted C₁₋₃ alkyl. In certain embodiments, R¹ is substituted C₁₋₃ alkyl. In certain embodiments, R¹ is unsubstituted C₁₋₃ alkyl. In certain embodiments, R¹ is methyl, ethyl, n-propyl, or iso-propyl. In certain embodiments, R¹ is n-butyl, sec-butyl, iso-butyl, or tert-butyl. In certain embodiments, R¹ is haloalkyl. In certain embodiments, R¹ is trihalomethyl. In certain embodiments, R¹ is trifluoromethyl. In certain embodiments, R¹ is halogen. In certain embodiments, R¹ is selected from the group consisting of chlorine (—Cl), bromine (—Br), fluorine (—F), and iodine (—I). In certain embodiments, R¹ is —I. In certain embodiments, R¹ is —F. In certain embodiments, R¹ is —Br. In certain embodiments, R¹ is —Cl.

### Group R²

As generally defined herein, each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c}, wherein each instance of R^{2a} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or an oxygen protecting group; each instance of R^{2b} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or a nitrogen protecting group; or optionally, two R^{2b} are taken together with the intervening atoms to form optionally substituted heterocyclyl or heteroaryl; and each instance of R^{2c} is independently hydrogen, optionally substituted alkenyl, optionally substituted alkynyl, optionally substituted aryl, optionally substituted heteroaryl, optionally substituted carbocyclyl, optionally substituted heterocyclyl, optionally substituted alkyl, optionally substituted acyl, or a sulfur protecting group.

In certain embodiments, at least one instance of R² is hydrogen. In certain embodiments, at least one instance of R² is halogen. In certain embodiments, at least one instance of R² is —CN. In certain embodiments, at least one instance of R² is —NO₂. In certain embodiments, at least one instance of R² is —N₃. In certain embodiments, at least one instance of R² is optionally substituted alkyl. In certain embodiments, at least one instance of R² is optionally substituted alkenyl. In certain embodiments, at least one instance of R² is optionally substituted alkynyl. In certain embodiments, at least one instance of R² is optionally substituted carbocyclyl. In certain embodiments, at least one instance of R² is optionally substituted aryl. In certain embodiments, at least one instance of R² is optionally substituted heterocyclyl. In certain embodiments, at least one instance of R² is optionally substituted heteroaryl. In certain embodiments, at least one instance of R² is optionally substituted acyl. In certain embodiments, at least one instance of R² is —OR^{2a}, wherein R^{2a} is as defined herein. In certain embodiments, at least one instance of R² is —N(R^{2b})₂, wherein R^{2b} is as defined herein. In certain embodiments, at least one instance of R² is or —SR^{2c}, wherein R^{2c} is as defined herein. In certain embodiments, at least one instance of R² is halogen. In certain embodiments, at least one instance of R² is selected from the group consisting of chlorine (—Cl), bromine (—Br), fluorine (—F), and iodine (—I). In certain embodiments, at least one instance of R² is —I. In certain embodiments, at least one instance of R² is —F. In certain embodiments, one instance of R² is —F. In certain embodiments, at least one instance of R² is —Br. In certain embodiments, at least one instance of R² is —Cl.

As generally defined herein, n is 0, 1, 2, 3, 4, or 5. In certain embodiments, n is 0. In certain embodiments, n is 1. In certain embodiments, n is 2. In certain embodiments, n is 3. In certain embodiments, n is 4. In certain embodiments, n is 5.

In certain embodiments, n is 1; and R² is halogen. In certain embodiments, n is 1; R² is halogen; and R² is para to the point of attachment of the tetrahydropyridine ring to the benzenoid ring. In certain embodiments, n is 1; and R² is selected from the group consisting of chlorine (—Cl), bromine (—Br), fluorine (—F), and iodine (—I). In certain embodiments, n is 1; and R² is —Cl. In certain embodiments, n is 1; and R² is —Br. In certain embodiments, n is 1; and R² is —F. In certain embodiments, n is 1; and R² is —I. In certain embodiments, n is 1; R² is —F; and R² is para to the point of attachment of the tetrahydropyridine ring to the benzenoid ring.

In certain embodiments, the moiety represented by the formula:

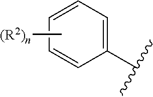

is of one of the following formulae:

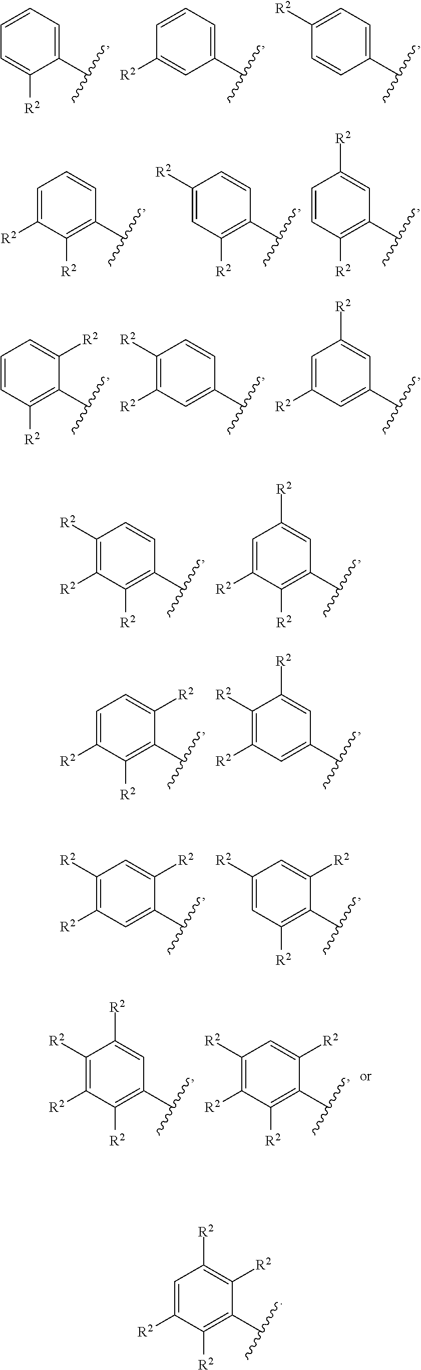

In certain embodiments, the moiety represented by the formula:

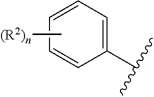

is of the formula:

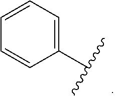 [SMILES: *c1ccccc1]

In certain embodiments, the moiety represented by the formula:

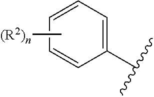

is of the formula:

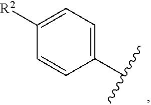

wherein R² is a halogen.

In certain embodiments, the moiety represented by the formula:

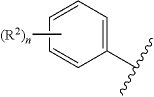

is of the formula:

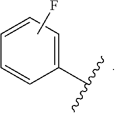

In certain embodiments, the moiety represented by the formula:

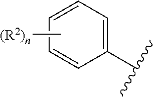

is of one of the following formulae:

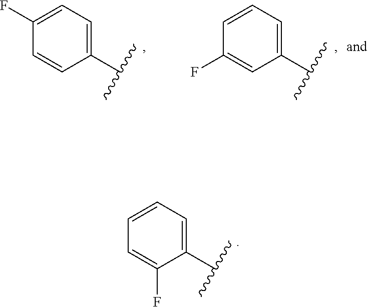 [SMILES: *c1ccc(F)cc1 ; *c1cccc(F)c1 ; *c1ccccc1F]

In certain embodiments, the moiety represented by the formula:

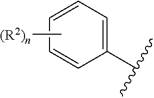

is of one of the following formula:

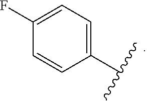 [SMILES: *c1ccc(F)cc1]

Groups R^{2a}, R^{2b}, and R^{2c}

As generally defined herein, each instance of R^{2a} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group. In certain embodiments, at least one instance of R^{2a} is hydrogen. In certain embodiments, at least one instance of R^{2a} is optionally substituted alkyl. In certain embodiments, at least one instance of R^{2a} is optionally substituted alkenyl. In certain embodiments, at least one instance of R^{2a} is optionally substituted alkynyl. In certain embodiments, at least one instance of R^{2a} is optionally substituted aryl. In certain embodiments, at least one instance of R^{2a} is optionally substituted heteroaryl. In certain embodiments, at least one instance of R^{2a} is optionally substituted carbocyclyl. In certain embodiments, at least one instance of R^{2a} is optionally substituted heterocyclyl. In certain embodiments, at least one instance of R^{2a} is optionally substituted acyl. In certain embodiments, at least one instance of R^{2a} is an oxygen protecting group.

As generally defined herein, each instance of R^{2b} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or nitrogen protecting group; or optionally, two R^{2b} are taken together with the intervening atoms to form optionally substituted heterocyclyl or heteroaryl. In certain embodiments, at least one instance of R^{2b} is hydrogen. In certain embodiments, at least one instance of R^{2b} is optionally substituted alkyl. In certain embodiments, at least one instance of R^{2b} is optionally substituted alkenyl. In certain embodiments, at least one instance of R^{2b} is optionally substituted alkynyl. In certain embodiments, at least one instance of R^{2b} is optionally substituted aryl. In certain embodiments, at least one instance of R^{2b} is optionally substituted heteroaryl. In certain embodiments, at least one instance of R^{2b} is optionally substituted carbocycyl. In certain embodiments, at least one instance of R^{2b} is optionally substituted heterocyclyl. In certain embodiments, at least one instance of R^{2b} is optionally substituted acyl. In certain embodiments, at least one instance of R^{2b} is a nitrogen protecting group. In certain embodiments, two R^{2b} are taken together with the intervening atoms to form optionally substituted heterocyclyl or heteroaryl. In certain embodiments, two R^{2b} are taken together with the intervening atoms to form optionally substituted heterocyclyl. In certain embodiments, two R^{2b} are taken together with the intervening atoms to form optionally substituted heteroaryl.

As generally defined herein, each instance of R^{2c} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group. In certain embodiments, at least one instance of R^{2c} is hydrogen. In certain embodiments, at least one instance of R^{2c} is optionally substituted alkyl. In certain embodiments, at least one instance of R^{2c} is optionally substituted alkenyl. In certain embodiments, at least one instance of R^{2c} is optionally substituted alkynyl. In certain embodiments, at least one instance of R^{2c} is optionally substituted aryl. In certain embodiments, at least one instance of R^{2c} is optionally substituted heteroaryl. In certain embodiments, at least one instance of R^{2c} is optionally substituted carbocyclyl. In certain embodiments, at least one instance of R^{2c} is optionally substituted heterocyclyl. In certain embodiments, at least one instance of R^{2c} is optionally substituted acyl. In certain embodiments, at least one instance of R^{2c} is a sulfur protecting group.

### Group R³

As general defined herein, R³ is optionally substituted, monocyclic or bicyclic carbocyclyl; optionally substituted, monocyclic or bicyclic aryl; optionally substituted, monocyclic or bicyclic heterocyclyl; or optionally substituted, monocyclic or bicyclic heteroaryl. In certain embodiments, R³ is optionally substituted, monocyclic or bicyclic carbocyclyl. In certain embodiments, R³ is optionally substituted, monocyclic carbocyclyl. In certain embodiments, R³ is substituted monocyclic carbocyclyl. In certain embodiments, R³ is unsubstituted monocyclic carbocyclyl. In certain embodiments, R³ is optionally substituted bicyclic carbocyclyl. In certain embodiments, R³ is substituted bicyclic carbocyclyl. In certain embodiments, R³ is unsubstituted bicyclic carbocyclyl.

In certain embodiments, R³ is optionally substituted, monocyclic or bicyclic aryl. In certain embodiments, R³ is optionally substituted monocyclic aryl. In certain embodiments, R³ is substituted monocyclic aryl. In certain embodiments, R³ is unsubstituted monocyclic aryl. In certain embodiments, R³ is optionally substituted bicyclic aryl. In certain embodiments, R³ is substituted bicyclic aryl. In certain embodiments, R³ is unsubstituted bicyclic aryl.

In certain embodiments, R³ is optionally substituted, monocyclic or bicyclic heterocyclyl. In certain embodiments, R³ is optionally substituted, monocyclic heterocyclyl. In certain embodiments, R³ is substituted monocyclic heterocyclyl. In certain embodiments, R³ is unsubstituted monocyclic heterocyclyl. In certain embodiments, R³ is optionally substituted bicyclic heterocyclyl. In certain embodiments, R³ is substituted bicyclic heterocyclyl. In certain embodiments, R³ is unsubstituted bicyclic heterocyclyl.

In certain embodiments, R³ is optionally substituted, monocyclic or bicyclic heteroaryl. In certain embodiments, R³ is optionally substituted monocyclic heteroaryl. In certain embodiments, R³ is substituted monocyclic heteroaryl. In certain embodiments, R³ is unsubstituted monocyclic heteroaryl. In certain embodiments, R³ is optionally substituted bicyclic heteroaryl. In certain embodiments, R³ is substituted bicyclic heteroaryl. In certain embodiments, R³ is unsubstituted bicyclic heteroaryl. In certain embodiments, R³ is of one of the following formulae:

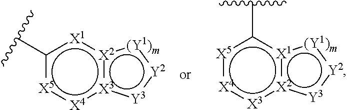

wherein X¹, X², X³, X⁴, X⁵, Y¹, Y², Y³, and m are as defined herein.

As generally defined herein, m is 1 or 2. In certain embodiments, m is 1. In certain embodiments, m is 2.

In certain embodiments, R³ is optionally substituted 5,6-bicyclic heteroaryl. In certain embodiments, R³ is substituted 5,6-bicyclic heteroaryl. In certain embodiments, R³ is unsubstituted 5,6-bicyclic heteroaryl. In certain embodiments, R³ is of one of the following formulae:

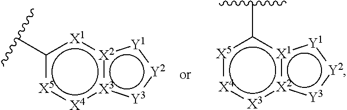

wherein X¹, X², X³, X⁴, X⁵, Y¹, Y², and Y³ are as defined herein.

In certain embodiments, R³ is optionally substituted 6,5-bicyclic heteroaryl, wherein the 6-membered ring contains at least one heteroatom selected from N, S, and O. In certain embodiments, R³ is optionally substituted 6,5-bicyclic heteroaryl, wherein the 6-membered ring contains at least one nitrogen atom. In certain embodiments, R³ is optionally substituted 6,5-bicyclic heteroaryl, wherein the 6-membered ring contains one nitrogen atom. In certain embodiments, R³ is optionally substituted 5,6-bicyclic heteroaryl, wherein the 5,6-bicyclic heteroaryl ring system comprises two nitrogen atoms. In certain embodiments, R³ is optionally substituted 5,6-bicyclic heteroaryl, wherein the 5,6-bicyclic heteroaryl ring system comprises one nitrogen atom in the 6-membered ring, and at least one nitrogen atom in the 5-membered ring. In certain embodiments, R³ is of one of the following formulae:

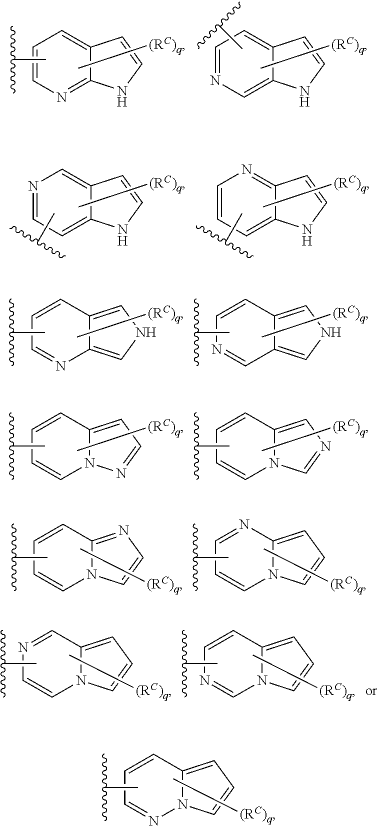

wherein R^{C} and q are as defined herein. In certain embodiments, R³ is of the following formula:

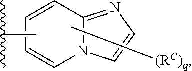

wherein R^{C} and q are as defined herein.

In certain embodiments, q is 0; and R³ is of one of the following formulae:

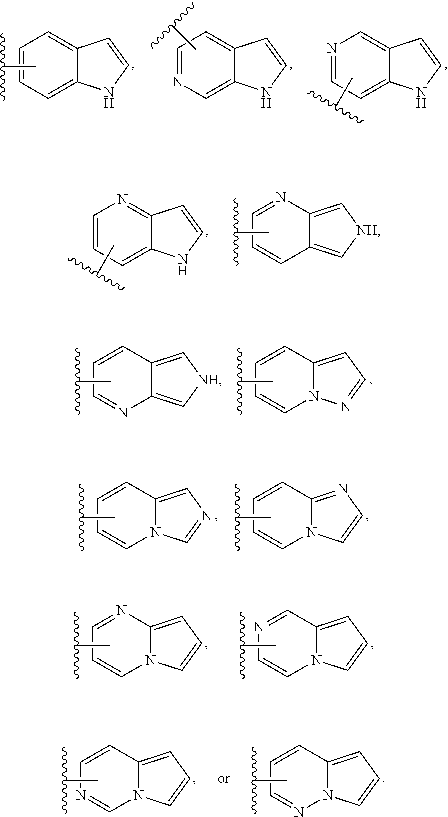

In certain embodiments, R³ is optionally substituted imidazo[1,2-a]pyridine. In certain embodiments, R³ is of one of the following formulae:

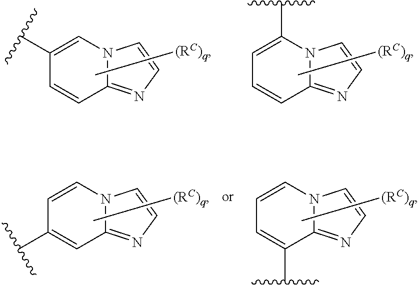

wherein R^{C} and q are as defined herein. In certain embodiments, q is 0; and R³ is one of the following formulae:

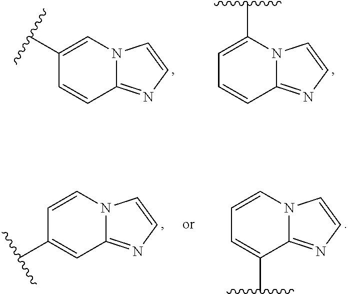 [SMILES: *c1ccc2nccn2c1 ; *c1cccc2nccn12 ; *c1ccn2ccnc2c1 ; *c1cccn2ccnc12]

In certain embodiments, R³ is of the following formula:

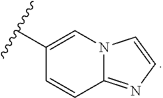 [SMILES: *c1ccc2nccn2c1]

In certain embodiments R³ is optionally substituted five-membered heteroaryl. In certain embodiments, R³ is optionally substituted six-membered heteroaryl. In certain embodiments, R³ is substituted six-membered heteroaryl. In certain embodiments, R³ is unsubstituted six-membered heteroaryl. In certain embodiments, R³ is optionally substituted six-membered heteroaryl, wherein the heteroaryl ring comprises at least one nitrogen atom. In certain embodiments, R³ is substituted six-membered heteroaryl, wherein the heteroaryl ring comprises at least one nitrogen atom. In certain embodiments, R³ is unsubstituted six-membered heteroaryl, wherein the heteroaryl ring comprises at least one nitrogen atom.

In certain embodiments, R³ is one of the following formulae:

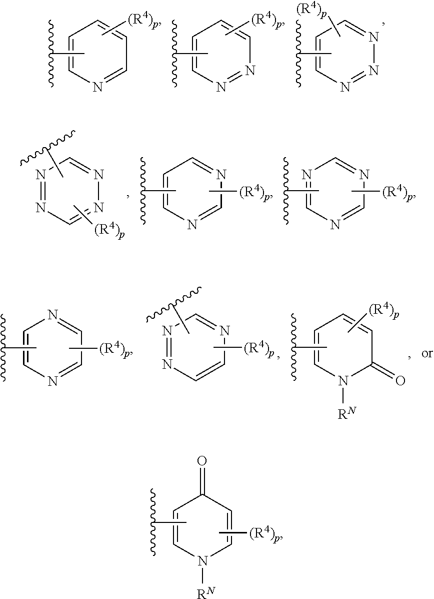

wherein R⁴, p, and R^{N} are as defined herein.

In certain embodiments, p is 0; and R³ is one of the following formulae:

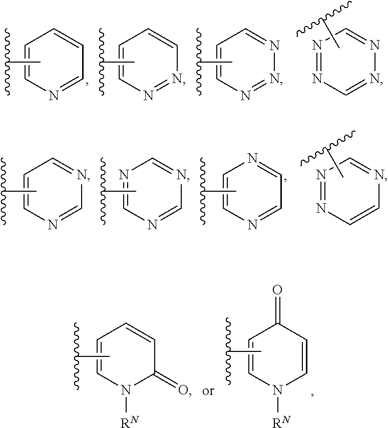

wherein R^{N} is as defined herein.

In certain embodiments, R³ is optionally substituted pyridone. In certain embodiments, R³ is optionally substituted 2-pyridone. In certain embodiments, R³ is one of the following formulae:

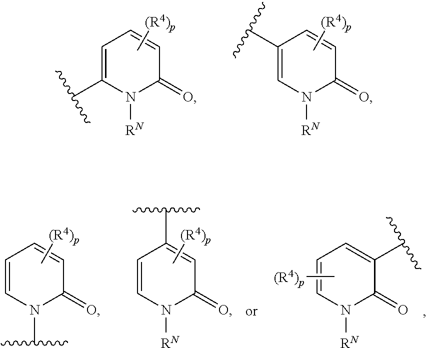

wherein R⁴, p, and R^{N} are as defined herein. In certain embodiments, p is 0; and R³ is one of the following formulae:

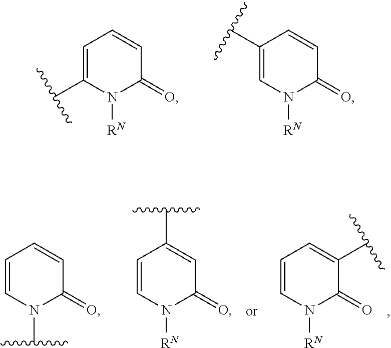

wherein R^{N} is as defined herein. In certain embodiments, R^{N} is hydrogen, and R³ is one of the following formulae:

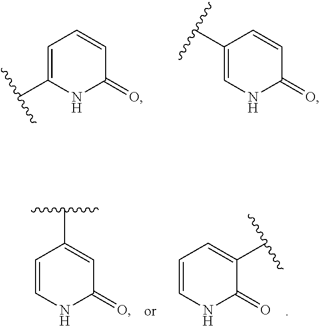 [SMILES: *c1cccc(=O)[nH]1 ; *c1ccc(=O)[nH]c1 ; *c1cc[nH]c(=O)c1 ; *c1ccc[nH]c1=O]

In certain embodiments, R³ is of the following formula:

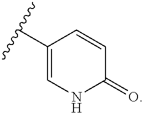 [SMILES: *c1ccc(=O)[nH]c1]

### Group R⁴

As generally defined herein, each instance of R⁴ is independently hydrogen, —CN, —NO₂, —N₃, optionally substituted alkyl, optionally substituted carbocyclyl, optionally substituted aryl, optionally substituted heterocyclyl, optionally substituted five-membered heteroaryl, optionally substituted acyl, —OR^{2a}, —N(R^{2b})₂, or —SR^{2c}, wherein R^{2a}, R^{2b}, and R^{2c} are as generally defined herein. In certain embodiments, at least one instance of R⁴ is hydrogen. In certain embodiments, at least one instance of R⁴ is —CN. In certain embodiments, at least one instance of R⁴ is —NO₂. In certain embodiments, at least one instance of R⁴ is —N₃. In certain embodiments, at least one instance of R⁴ is optionally substituted alkyl. In certain embodiments, at least one instance of R⁴ is optionally substituted carbocyclyl. In certain embodiments, at least one instance of R⁴ is optionally substituted aryl. In certain embodiments, at least one instance of R⁴ is optionally substituted heterocyclyl. In certain embodiments, at least one instance of R⁴ is optionally substituted five-membered heteroaryl. In certain embodiments, at least one instance of R⁴ is optionally substituted acyl. In certain embodiments, at least one instance of R⁴ is —OR^{2a}, wherein R^{2a} is as defined herein. In certain embodiments, at least one instance of R⁴ is —N(R^{2b})₂, wherein R^{2b} is as defined herein. In certain embodiments, at least one instance of R⁴ is —SR^{2c}, wherein R^{2c} is as defined herein. In certain embodiments, all instances of R⁴ are hydrogen.

As generally defined herein, p is 0, 1, 2, or 3. In certain embodiments, p is 0. In certain embodiments, p is 1. In certain embodiments, p is 2. In certain embodiments, p is 3.

X¹, X², X³, X⁴, X⁵, Y¹, Y², and Y³

As generally defined herein, each of X¹, X², X³, X⁴, X⁵, Y¹, Y², and Y³ is independently C, CR^{C}, N, NR^{N}, S, or O, as valency permits; provided that at least one of X¹, X², X³, X⁴, or X⁵ is N, S or O. In certain embodiments, each of X¹, X², X³, X⁴, X⁵, Y², and Y³ is independently C, CR^{C}, N, NR^{N}, S, or O, as valency permits; provided that at least one of X¹, X², X³, X⁴, or X⁵ is N. In certain embodiments, each of X¹, X², X³, X⁴, X⁵, Y¹, Y² and Y³ is independently C, CR^{C}, N, NR^{N}, S, or O, as valency permits; provided that at least one of X¹, X², X³, X⁴, or X⁵ is N; and provided at least one instance of Y¹, Y², and Y³ is N or NR^{N}, as valency permits.

In certain embodiments, X¹ is C. In certain embodiments, X¹ is CR^{C}. In certain embodiments, X¹ is N. In certain embodiments, X¹ is NR^{N}. In certain embodiments, X¹ is S. In certain embodiments, X¹ is O.

In certain embodiments, X² is C. In certain embodiments, X² is CR^{C}. In certain embodiments, X² is N. In certain embodiments, X² is NR^{N}. In certain embodiments, X² is S. In certain embodiments, X² is O.

In certain embodiments, X³ is C. In certain embodiments, X³ is CR^{C}. In certain embodiments, X³ is N. In certain embodiments, X³ is NR^{N}. In certain embodiments, X³ is S. In certain embodiments, X³ is O.

In certain embodiments, X⁴ is C. In certain embodiments, X⁴ is CR^{C}. In certain embodiments, X⁴ is N. In certain embodiments, X⁴ is NR^{N}. In certain embodiments, X⁴ is S. In certain embodiments, X⁴ is O.

In certain embodiments, X⁵ is C. In certain embodiments, X⁵ is CR^{C}. In certain embodiments, X⁵ is N. In certain embodiments, X⁵ is NR^{N}. In certain embodiments, X⁵ is S. In certain embodiments, X⁵ is O.

In certain embodiments, Y¹ is C. In certain embodiments, Y¹ is CR^{C}. In certain embodiments, Y¹ is N. In certain embodiments, Y¹ is NR^{N}. In certain embodiments, Y¹ is S. In certain embodiments, Y¹ is O.

In certain embodiments, Y² is C. In certain embodiments, Y² is CR^{C}. In certain embodiments, Y² is N. In certain embodiments, Y² is NR^{N}. In certain embodiments, Y² is S. In certain embodiments, Y² is O.

In certain embodiments, Y³ is C. In certain embodiments, Y³ is CR^{C}. In certain embodiments, Y³ is N. In certain embodiments, Y³ is NR^{N}. In certain embodiments, Y³ is S. In certain embodiments, Y³ is O.

In certain embodiments, at least one of X¹, X², X³, X⁴, or X⁵ is N. In certain embodiments, one of X¹, X², X³, X⁴, or X⁵ is N; and the rest are C or CR^{C}, as valency permits. In certain embodiments, at least one of Y¹, Y², or Y³ is N or NR^{N}, as valency permits. In certain embodiments, one of Y¹, Y², or Y³ is N or NR^{N}, as valency permits; and the rest are CR^{C}. In certain embodiments, at least one of X¹, X², X³, X⁴, or X⁵ is N; and at least one of Y¹, Y², or Y³ is N or NR^{N}, as valency permits. In certain embodiments, one of X¹, X², X³, X⁴, or X⁵ is N; and the rest are C or CR^{C}, as valency permits; and one of Y¹, Y², or Y³ is N or NR^{N}, as valency permits; and the rest are CR^{C}. In certain embodiments, X² is N; X³ is C; X¹, X⁴, and X⁵ are CR^{C}; Y³ is N; and Y¹ and Y² are CR^{C}. For example, in certain embodiments, the moiety represented by the formula:

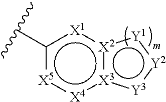

is of the formula:

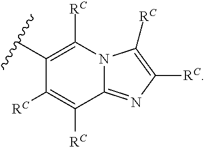

Groups R^{C} and R^{N}

As generally defined herein, each instance of R^{C} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c}, wherein R^{2a}, R^{2b}, and R^{2c} are as defined herein. In certain embodiments, at least one instance of R^{C} is hydrogen. In certain embodiments, at least one instance of R^{C} is halogen. In certain embodiments, at least one instance of R^{C} is —CN. In certain embodiments, at least one instance of R^{C} is —NO₂. In certain embodiments, at least one instance of R^{C} is —N₃. In certain embodiments, at least one instance of R^{C} is optionally substituted alkyl. In certain embodiments, at least one instance of R^{C} is optionally substituted alkenyl. In certain embodiments, at least one instance of R^{C} is optionally substituted alkynyl. In certain embodiments, at least one instance of R^{C} is optionally substituted carbocyclyl. In certain embodiments, at least one instance of R^{C} is optionally substituted aryl. In certain embodiments, at least one instance of R^{C} is optionally substituted heterocyclyl. In certain embodiments, at least one instance of R^{C} is optionally substituted heteroaryl. In certain embodiments, at least one instance of R^{C} is optionally substituted acyl. In certain embodiments, at least one instance of R^{C} is —OR^{2a}, wherein R^{2a} is as defined herein. In certain embodiments, at least one instance of R^{C} is —N(R^{2b})₂, wherein R^{2b} is as defined herein. In certain embodiments, at least one instance of R^{C} is —SR^{2c}, wherein R^{2c} is as defined herein.

As generally defined herein, each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group. In certain embodiments, at least one instance of R^{N} is hydrogen. In certain embodiments, at least one instance of R^{N} is optionally substituted alkyl. In certain embodiments, at least one instance of R^{N} is optionally substituted alkenyl. In certain embodiments, at least one instance of R^{N} is optionally substituted alkynyl. In certain embodiments, at least one instance of R^{N} is optionally substituted aryl. In certain embodiments, at least one instance of R^{N} is optionally substituted heteroaryl. In certain embodiments, at least one instance of R^{N} is optionally substituted carbocyclyl. In certain embodiments, at least one instance of R^{N} is optionally substituted heterocyclyl. In certain embodiments, at least one instance of R^{N} is optionally substituted acyl. In certain embodiments, at least one instance of R^{N} is a nitrogen protecting group. In certain embodiments, at least one instance of R^{N} is optionally substituted C₁₋₆ alkyl. In certain embodiments, at least one instance of R^{N} is substituted C₁₋₆ alkyl. In certain embodiments, at least one instance of R^{N} is unsubstituted C₁₋₆ alkyl. In certain embodiments, at least one instance of R^{N} is optionally substituted C₁₋₃ alkyl. In certain embodiments, at least one instance of R^{N} is substituted C₁₋₃ alkyl. In certain embodiments, at least one instance of R^{N} is unsubstituted C₁₋₃ alkyl. In certain embodiments, at least one instance of R^{N} is methyl, ethyl, n-propyl, iso-propyl, n-butyl, iso-butyl, sec-butyl, or tert-butyl. In certain embodiments, at least one instance of R^{N} is methyl.

### Pharmaceutical Compositions, Kits, and Administration

The present invention provides pharmaceutical compositions comprising a compound of Formula (I), (II), or (III), or a pharmaceutically acceptable salt, hydrate, solvate, polymorph, co-crystal, tautomer, stereoisomer, isotopically labeled derivative, or prodrug thereof, and optionally a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease in a subject in need thereof. In certain embodiments, the effective amount is an amount effective for treating a proliferative disease in a subject in need thereof. In certain embodiments, the effective amount (e.g., prophylactically effective amount) is an amount effective for preventing a proliferative disease in a subject in need thereof. Exemplary proliferative diseases include, but are not limited to, cancers, benign neoplasms, diseases associated with angiogenesis, inflammatory diseases, and autoimmune diseases. In certain embodiments, the effective amount is an amount effective for treating cancer in a subject in need thereof. In certain embodiments, the cancer is pancreatic cancer. In certain embodiments, the cancer is lung cancer (e.g., squamous cell carcinoma). In certain embodiments, the cancer is breast cancer. In certain embodiments, the proliferative disease is an inflammatory disease (e.g., rheumatoid arthritis). In certain embodiments, the effective amount is an amount effective for reducing the risk of developing a disease (e.g., a proliferative disease such as cancer, inflammatory diseases) in a subject in need thereof. In certain embodiments, the effective amount is an amount effective for preventing a proliferative disease in a subject in need thereof. In certain embodiments, the effective amount is an amount effective for preventing a recurrence of cancer (e.g., breast cancer, lung cancer (e.g., squamous cell carcinoma), pancreatic cancer) in a subject in need thereof.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Formulations suitable for topical administration include, but are not limited to, liquid and/or semi liquid preparations such as liniments, lotions, oil-in-water and/or water-in-oil emulsions such as creams, ointments, and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets, and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition of the invention can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive of a compound described herein. In certain embodiments, a dose described herein includes independently between 10 mg and 30 mg, inclusive of a compound described herein. In certain embodiments, a dose described herein includes independently between 30 mg and 100 mg inclusive of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). For example, in certain embodiments, a kit provided herein comprises a compound of Formula (I), (II), or (III), or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., a proliferative disease such as cancer or an inflammatory disease) in a subject in need thereof. In certain embodiments, the kits are useful for preventing a disease (e.g., a proliferative disease) in a subject in need thereof. In certain embodiments, the kits are useful for reducing the risk of developing a disease (e.g., a proliferative disease) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease such as cancer) in a subject in need thereof. In certain embodiments, the kits and instructions provide for preventing a disease (e.g., proliferative disease such as cancer) in a subject in need thereof. In certain embodiments, the kits and instructions provide for reducing the risk of developing a disease (e.g., proliferative disease such as cancer) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Methods of Treatment and Uses

The present invention also provides methods of treating and/or preventing diseases and conditions. The methods of treating and/or preventing diseases and conditions described herein comprise administering to a subject in need thereof a compound of Formula (I), (II), or (III), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the disease is a proliferative disease, wherein “proliferative disease” is as defined herein. Examples of proliferative diseases include, but are not limited to, benign neoplasms, diseases associated with angiogenesis, inflammatory diseases, and autoimmune diseases. In certain embodiments, the disease is an inflammatory disease. In certain embodiments, the disease is arthritis (e.g., rheumatoid arthritis, osteoarthritis). In certain embodiments, the disease is cancer, wherein “cancer” is as defined herein. All cancers disclosed herein or known in the art are contemplated as being within the scope of the invention. In certain embodiments, the cancer is pancreatic cancer. In certain embodiments, the cancer is lung cancer. In certain embodiments, the cancer is breast cancer. In certain embodiments, the cancer is squamous cell carcinoma. In a particular embodiment, the cancer is lung squamous cell carcinoma. In certain embodiments, the cancer is a cancer which is characterized by an amplification of chromosome 3q26. Certain cancers with an amplification of chromosome 3q26 include, but are not limited to, head cancer, neck cancer, esophageal cancer, and ovarian cancer. In certain embodiments, the cancer is head cancer. In certain embodiments, the cancer is neck cancer. In certain embodiments, the cancer is esophageal cancer. In certain embodiments, the cancer is neck cancer.

In certain embodiments, the disease to be treated and/or prevented is a disease associated with hedgehog acyltransferase (Hhat). For example, in certain embodiments, the disease is a cancer which is reliant on the Hhat signaling pathway. In certain embodiments, the disease to be treated and/or prevented is a disease associated with a Hedgehog protein (e.g., Shh, Dhh, Ihh). In certain embodiments, the disease to be treated and/or prevented is a disease associated with Sonic hedgehog (Shh). In certain embodiments, the disease to be treated and/or prevented is associated with aberrant (e.g., increased) Hedgehog (e.g., Shh) signaling. In certain embodiments, the disease to be treated and/or prevented is associated with aberrant Shh signaling. In certain embodiments, the disease to be treated and/or prevented is associated with overexpression of Sonic hedgehog (Shh). In certain embodiments, the disease to be treated and/or prevented is associated with aberrant (e.g., increased) Shh signaling. In certain embodiments, the disease to be treated and/or prevented is a proliferative disease associated with overexpression of Shh. In certain embodiments, the disease to be treated is cancer associated with overexpression of Shh. In certain embodiments, the disease to be treated and/or prevented is a proliferative disease associated with aberrant (e.g., increased) Shh signaling. In certain embodiments, the disease to be treated is cancer associated with aberrant (e.g., increased) Shh signaling. Cancers known to be associated with aberrant Shh signaling (e.g., increased Shh signaling) include, but are not limited to, pancreatic cancer, breast cancer, and lung cancer (e.g., lung squamous cell carcinoma).

As demonstrated and described herein, the compounds of the present invention inhibit hedgehog acyltransferase (Hhat). Without wishing to be bound by any particular theory, inhibition of Hhat can block acylation of Hedgehog proteins (e.g., Shh, Dhh, Ihh) and mitigate hedgehog signaling. For example, Hhat inhibition can block palmitoylation of Sonic hedgehog (Shh) and mitigate Shh signaling. Hedgehog acyltransferase may also be involved in non-canonical pathways, and therefore other signaling pathways could be affected by Hhat inhibition.

In one aspect, the present invention provides methods of inhibiting Hedgehog acyltransferase with a compound of Formula (I), (II), or (III), or a pharmaceutically acceptable salt, solvate, hydrate, polymorph, co-crystal, tautomer, stereoisomer, isotopically labeled derivative, and prodrug thereof, or a pharmaceutical composition thereof. In certain embodiments, provided herein is a method of inhibiting Hhat in a subject, the method comprising administering to the subject an effective amount (e.g., therapeutically effective amount) of a compound described herein, or a salt, solvate, hydrate, polymorph, co-crystal, tautomer, stereoisomer, isotopically labeled derivative, or prodrug thereof, or a pharmaceutical composition thereof. In certain embodiments, the present invention provides methods of inhibiting Hhat in a biological sample, the methods comprising contacting the biological sample with an effective amount of a compound described herein, or a salt, solvate, hydrate, polymorph, co-crystal, tautomer, stereoisomer, isotopically labeled derivative, or prodrug thereof, or a pharmaceutical composition thereof. In certain embodiments, the present invention provides methods of inhibiting Hhat in a cell, the methods comprising contacting the biological sample with an effective amount of a compound described herein, or a salt, solvate, hydrate, polymorph, co-crystal, tautomer, stereoisomer, isotopically labeled derivative, or prodrug thereof, or a pharmaceutical composition thereof.

In certain embodiments, the method of inhibiting Hhat comprises contacting a Hhat protein with a compound of Formula (I), (II), or (III), or a salt, solvate, hydrate, polymorph, co-crystal, tautomer, stereoisomer, isotopically labeled derivative, or prodrug thereof, or a pharmaceutical composition thereof. In certain embodiments, the step of contacting the Hhat occurs in vivo. In certain embodiments, the step of contacting the Hhat occurs in vitro. The Hhat may be purified or crude, and may be present in a cell, tissue, biological sample, or subject. Inhibition of Hhat does not require that all of the protein be contacted by an inhibitor at once. Exemplary levels of inhibition of Hhat include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

Also provided herein is a method of mitigating hedgehog signaling (e.g., Shh signaling, Dhh signaling, Ihh signaling) in a cell and/or a subject with a compound of Formula (I), (II), or (III), or a salt, solvate, hydrate, polymorph, co-crystal, tautomer, stereoisomer, isotopically labeled derivative, or prodrug thereof, or a composition thereof. For example, provided herein is a method of mitigating Sonic hedgehog (Shh) signaling in a cell and/or a subject with a compound of Formula (I), (II), or (III), or a salt, solvate, hydrate, polymorph, co-crystal, tautomer, stereoisomer, isotopically labeled derivative, or prodrug thereof, or a composition thereof. Without wishing to be bound by any particular theory, inhibition of Hhat inhibits post-translational modification (e.g., palmitoylation) of hedgehog proteins (e.g., Shh), which mitigates hedgehog signaling (e.g., Shh signaling). Mitigating Shh signaling is beneficial in the prevention and/or treatment of proliferative diseases such as cancer. In certain embodiments, the method of mitigating hedgehog signaling (e.g., Shh signaling) in a cell and/or a subject comprises contacting the cell or subject with an effective amount of a compound of Formula (I), (II), or (III), or a salt, solvate, hydrate, polymorph, co-crystal, tautomer, stereoisomer, isotopically labeled derivative, or prodrug thereof, or a composition thereof.

In yet another aspect, provided herein are methods of inducing apoptosis using a compound of Formula (I), (II), or (III), or a salt, solvate, hydrate, polymorph, co-crystal, tautomer, stereoisomer, isotopically labeled derivative, or prodrug thereof, or a pharmaceutical composition thereof. “Apoptosis” is defined herein. In certain embodiments, the method of inducing apoptosis provided herein comprises contacting a cell with a Hhat inhibitor. Thus, the method of inducing apoptosis can comprise contacting a cell with a compound of Formula (I), (II), or (III), or a salt, solvate, hydrate, polymorph, co-crystal, tautomer, stereoisomer, isotopically labeled derivative, or prodrug thereof, or a pharmaceutical composition thereof. In certain embodiments, the inventive compound induces apoptosis in vivo. In certain embodiments, the inventive compound induces apoptosis in vitro.

In certain embodiments, the methods described herein comprise administering to a subject a therapeutically effective amount compound of Formula (I), (II), or (III), or a pharmaceutically acceptable salt, solvate, hydrate, polymorph, co-crystal, tautomer, stereoisomer, isotopically labeled derivative, or prodrug thereof, or a pharmaceutical composition thereof, wherein “therapeutically effective amount” is as defined herein. In certain embodiments, the methods described herein include contacting a biological sample with an effective amount of a compound of Formula (I), (II), or (III), or a pharmaceutically acceptable salt, solvate, hydrate, polymorph, co-crystal, tautomer, stereoisomer, isotopically labeled derivative, or prodrug thereof, or a pharmaceutical composition thereof.

A compound or composition provided herein may be administered concurrently with, prior to, or subsequent to, one or more additional therapeutically active agents. In general, each agent will be administered at a dose and/or on a time schedule determined for that agent. It will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. In certain embodiments, the additional therapeutic agent is an anti-proliferative agent, wherein “anti-proliferative” agent is as defined herein. In certain embodiments, the additional therapeutic agent is an anti-cancer agent, wherein “anti-cancer” agent is as defined herein. In certain embodiments, the additional therapeutic agent is an Hhat inhibitor.

In certain embodiments, the compounds or pharmaceutical compositions described herein can be administered in combination with an anti-cancer therapy including, but not limited to, surgery, radiation therapy, transplantation (e.g., stem cell transplantation, bone marrow transplantation), immunotherapy, and chemotherapy.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

In certain embodiments, the provided methods comprise contacting a cell with an effective amount of a compound or a pharmaceutical composition as described herein. The cell may be contacted in vitro or in vivo. In certain embodiments, the contacting is in vivo. In certain embodiments, the contacting is in vitro. In certain embodiments, the cell is a cancer cell. In certain embodiments, the cell is an isogenic cancer cell. In certain embodiments, the cell is a pancreatic cancer cell. In certain embodiments, the cell is a breast cancer cell. In certain embodiments, the cell is a lung cancer cell. In certain embodiments, the cell is a human cell. In certain embodiments, the cell is non-human animal cell.

The present invention provides uses of the inventive compounds (e.g., compounds of Formulae (I), (II), (III)) and pharmaceutically acceptable salts, solvates, hydrates, polymorphs, co-crystals, tautomers, stereoisomers, prodrugs, and pharmaceutical compositions thereof, for the treatment and/or prevention of diseases described herein.

The present invention also provides uses of the inventive compounds (e.g., compounds of Formulae (I), (II), (III)) and pharmaceutically acceptable salts, solvates, hydrates, polymorphs, co-crystals, tautomers, stereoisomers, prodrugs, and pharmaceutical compositions thereof, in the manufacture of medicaments for the treatment and/or prevention of diseases described herein.

The present invention also provides compounds (e.g., compounds of Formulae (I), (II), (III)) and pharmaceutically acceptable salts, solvates, hydrates, polymorphs, co-crystals, tautomers, stereoisomers, prodrugs, and pharmaceutical compositions thereof, for use in treating and/or preventing diseases discussed herein.

### EXAMPLES

In order that the invention described herein may be more fully understood, the following examples are set forth. It should be understood that these examples are for illustrative purposes only and are not to be construed as limiting this invention in any manner.

### Synthetic Procedures

### General Information

Solvents and reagents were purchased from VWR or sigma Aldrich. All reactions involving air- or moisture-sensitive compounds were performed under nitrogen atmosphere using dried glassware. ¹H and ¹³C NMR spectra were recorded at 500 MHz and 125 Mz respectively, on a Bruker Advance III HD 500 MHz NMR spectrometer equipped with at TCI cryogenic probe with enhanced ¹H and ¹³C detection. All data was collected at 298 K, signals were reported in ppm, internally referenced for ¹H and 13C to chloroform signal at 7.26 ppm or 77.0 ppm; to DMSO signal at 2.50 ppm or 39.5 ppm, or TMS at 0 ppm. Chemical shifts are reported in parts per million (ppm) and the coupling constants (J) are expressed in hertz (Hz). Splitting patterns are designated as follows: s, singlet; d, doublet; t, triplet; m, multiplet; dd, doublet of doublets; ddd, double of doublets of doublets; dt, doublet of triplets. Flash chromatography purifications were performed on CombiFlash Rf (TELEDYNE ISCO) as the stationary phase. Melting points were determined on a MP50 Melting Point System (METTLER TOLEDO). Purity for all tested compounds was determined through high-performance liquid chromatography and all compounds were found to be >95% pure.

### Example 1

### N-(2-(thiophen-2-yl)ethyl)benzamide

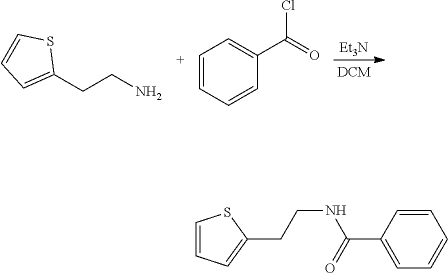 [SMILES: "Et3N" ; NCCc1cccs1 ; O=C(Cl)c1ccccc1 ; O=C(NCCc1cccs1)c1ccccc1]

To a solution of 2-(thiophen-2-yl)ethan-1-amine (1.27 g, 10.00 mmol) and DIPEA (2.58 g, 20.00 mmol) in CH₂Cl₂ (20 mL) at 0° C. was added benzoyl chloride (1.41 g, 10.00 mmol) dropwise. The mixture was stirred at 20° C. for 15 mins. The mixture was diluted with CH₂Cl₂ (30 mL) and washed with sat.NaHCO₃ aq. (30 mL). The organic layer was washed by 1N HCl (30 mL) and brine (30 mL), dried over Na₂SO₄ and filtered, concentrated in vacuo to give N-(2-(thiophen-2-yl)ethyl)benzamide (2.25 g, 9.73 mmol, 97% yield) as an off-white solid. 1H NMR: (400 MHz, CDCl₃) δ 7.73 (d, J=8.0 Hz, 2H), 7.49 (d, J=8.0 Hz, 1H), 7.42 (t, J=8.0 Hz, 2H), 7.20 (d, J=4.0 Hz, 1H), 6.98 (t, J=4.0 Hz, 1H), 6.90 (s, 1H), 6.32 (brs, 1H), 3.76 (q, J=6.4 Hz, 2H), 3.19 (t, J=6.4 Hz, 2H).

### 4-phenyl-6,7-dihydrothieno[3,2-c]pyridine

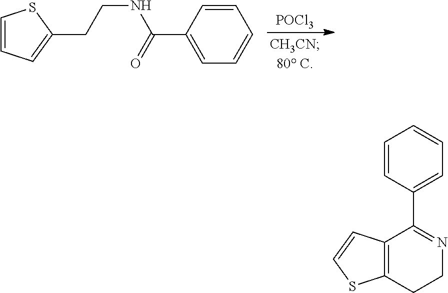

To a solution of N-(2-(thiophen-2-yl)ethyl)benzamide (1.00 g, 4.32 mmol) in CH₃CN (20 mL) was added POCl₃ (3.31 g, 21.60 mmol) in one portion. The mixture was stirred at 80° C. for 1.5 h. The mixture was concentrated in vacuo and the residue was diluted with CH₂Cl₂ (30 mL) and quenched with sat.NaHCO₃ aq. (30 mL). The organic layer was dried over Na₂SO₄ and filtered, concentrated in vacuo to give the crude 4-phenyl-6,7-dihydrothieno[3,2-c]pyridine (1.00 g) which was used directly for next step without further purification.

### 4-phenyl-4,5,6,7-tetrahydrothieno[3,2-c]pyridine

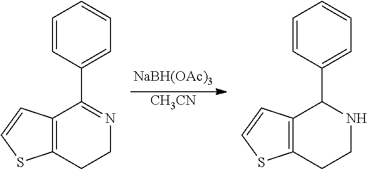

To a solution of 4-phenyl-6,7-dihydrothieno[3,2-c]pyridine (1.00 g) in CH₃CN (10.00 mL) at 20° C. was added NaBH(OAc)₃ (1.99 g, 9.38 mmol) in portion wise. The mixture was stirred at 20° C. for 12 h. The mixture was concentrated in vacuo and the residue was diluted with EtOAc (30 mL) and washed by 1N HCl (30 mL). The aqueous layer was separated and basified with sat.Na₂CO₃ aq. (25 mL). The mixture was extracted with EtOAc (25 mL×2). The combined organic layer was dried over Na₂SO₄, filtered and concentrated in vacuo to give 4-phenyl-4,5,6,7-tetrahydrothieno[3,2-c]pyridine (350.00 mg, 33% yield over two steps) as a white solid. LCMS: RT=0.748 min; m/z 216.2 (M+H)⁺. ¹H NMR: (CDCl₃, 400 MHz) δ 7.37-7.26 (m, 5H), 7.11 (d, J=5.2 Hz, 1H), 6.42 (d, J=5.2 Hz, 1H), 5.02 (s, 1 H), 3.31-3.26 (m, 1H), 3.01-2.93 (m, 2H), 2.91-2.86 (m, 1H).

### 5-(4-phenyl-4,5,6,7-tetrahydrothieno[3,2-c]pyridine-5-carbonyl)pyridin-2(1H)-one

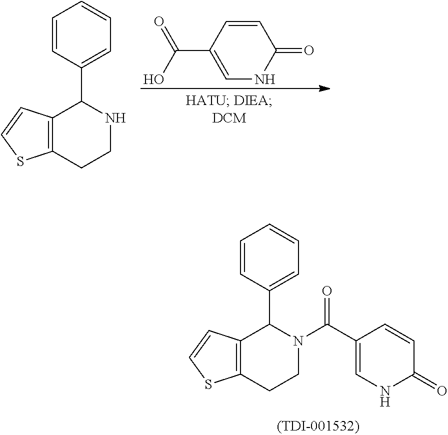 [SMILES: c1ccc(C2NCCc3sccc32)cc1 ; O=C(O)c1ccc(=O)[nH]c1 ; O=C(c1ccc(=O)[nH]c1)N1CCc2sccc2C1c1ccccc1]

To a solution of 4-phenyl-4,5,6,7-tetrahydrothieno[3,2-c]pyridine (100.00 mg, 464.45 μmol) and 6-oxo-1,6-dihydropyridine-3-carboxylic acid (64.61 mg, 464.45 μmol) in CH₂Cl₂ (15 mL) was added HATU (176.60 mg, 464.45 μmol) and DIPEA (180.08 mg, 1.39 mmol). The mixture was stirred at 20° C. for 0.5 h. The mixture was filtered and the filter cake was triturated by CH₃CN (10 mL) to give 5-(4-phenyl-4,5,6,7-tetrahydrothieno[3,2-c]pyridine-5-carbonyl)pyridin-2(1H)-one (35.00 mg, 101.13 μmol, 22% yield) as an off-white solid. LCMS: RT=1.842 min; m/z 337.2 (M+H)⁺. ¹H NMR: (CDCl₃, 400 MHz) δ 12.25 (brs, 1H), 7.60-7.55 (m, 2H), 7.36-7.21 (m, 5H), 7.21 (d, J=5.2 Hz, 1H), 6.76 (d, J=5.2 Hz, 1H), 6.60 (d, J=9.2 Hz, 1H), 4.14-4.08 (m, 1H), 3.43 (t, J=9.6 Hz, 1H), 3.11-2.83 (m, 2H).

### Example 2

### 1-methyl-5-(4-phenyl-4,5,6,7-tetrahydrothieno[3,2-c]pyridine-5-carbonyl)pyridin-2(1H)-one

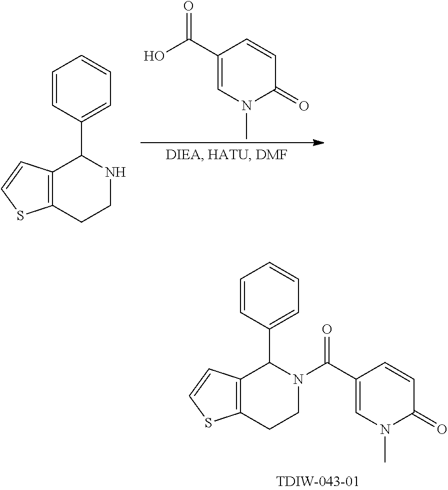 [SMILES: c1ccc(C2NCCc3sccc32)cc1 ; Cn1cc(C(=O)O)ccc1=O ; Cn1cc(C(=O)N2CCc3sccc3C2c2ccccc2)ccc1=O]

To a solution of 4-phenyl-4,5,6,7-tetrahydrothieno[3,2-c]pyridine (100 mg, 653.00 μmol) in DMF (10 mL) was added HATU (372 mg, 979.50 μmol) and DIPEA (253 mg, 1.96 mmol) at 0° C. and stirred for 30 min at 0° C. Then 1-methyl-6-oxo-1,6-dihydropyridine-3-carboxylic acid (154 mg, 718.30 μmol) was added at 0° C. in one portion and the mixture reaction was stirred at room temperature for 2 h. The mixture was concentrated in vacuo. The residue was dissolved in H₂O (10 mL) and EtOAc (10 mL) and extracted with EtOAc (10 mL*2). The combined organic layers were washed with brine (10 mL*2), dried over anhydrous Na₂SO₄, filtered and concentrated in vacuo. The residue was purified by prep-HPLC (Phenomenex Synergi C18 150*30 mm*4 um, Flow Rate: 28 (ml/min), 0.05% HCl—CH₃CN, 35% CH₃CN to 55% CH₃CN over 9 min) to give 1-methyl-5-(4-phenyl-4,5,6,7-tetrahydrothieno[3,2-c]pyridine-5-carbonyl)pyridin-2(1H)-one (98.00 mg, 279.66 μmol, 43% yield) as a white solid. LCMS: RT=1.792 min, m/z 351.1 (M+H)⁺. ¹H NMR (CD₃OD, 400 MHz) δ 7.89 (d, J=2.0 Hz, 1H), 7.56-7.53 (m, 1H), 7.32-7.28 (m, 6H), 6.73-6.70 (m, 2 H), 6.56 (d, J=9.2 Hz, 1H), 4.09 (s, 1H), 3.56 (s, 3H), 3.46-3.45 (m, 1H), 3.10-3.07 (m, 1H), 2.97-2.96 (m, 1H).

### Example 3

### tert-butyl 4-phenyl-6,7-dihydrothieno[3,2-c]pyridine-5(4H)-carboxylate

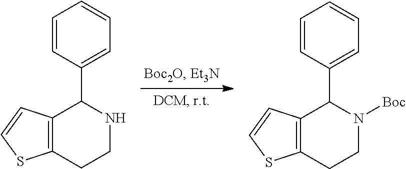 [SMILES: c1ccc(C2NCCc3sccc32)cc1 ; CC(C)(C)OC(=O)N1CCc2sccc2C1c1ccccc1]

To a solution of 4-phenyl-4,5,6,7-tetrahydrothieno[3,2-c]pyridine (1.5 g, 6.97 mmol) in CH₂Cl₂ (20 mL) was added Boc₂O (2.3 g, 10.45 mmol) and NEt₃ (2.1 g, 20.91 mmol) at 20° C. The reaction mixture was stirred for 2 h at 20° C. The mixture was concentrated in vacuo and the residue was diluted with EtOAc (50 mL). N, N-dimethylenediamine (1 g) was added to the mixture to remove excess Boc₂O and stirred for 30 mins. Then the organic phase was washed with 1N HCl (20 mL*4) and then brine (20 mL*2), dried over anhydrous Na₂SO₄, filtered and concentrated in vacuo to give tert-butyl 4-phenyl-6,7-dihydrothieno[3,2-c]pyridine-5(4H)-carboxylate (1.9 g, 86% yield) as a pale yellow solid. LCMS: RT=0.946 min, m/z 260.1 (M-t-butyl+H)⁺.

### tert-butyl 2-chloro-4-phenyl-6,7-dihydrothieno[3,2-c]pyridine-5(4H)-carboxylate

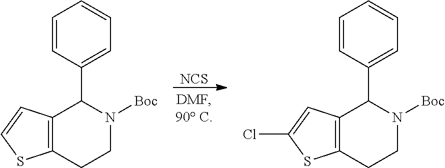 [SMILES: CC(C)(C)OC(=O)N1CCc2sccc2C1c1ccccc1 ; "NCS" ; CC(C)(C)OC(=O)N1CCc2sc(Cl)cc2C1c1ccccc1]

To a solution of tert-butyl 4-phenyl-6,7-dihydrothieno[3,2-c]pyridine-5(4H)-carboxylate (1.5 g, 4.71 mmol) in DMF (20 mL) was added NCS (692 mg, 5.18 mmol) in one portion and the reaction mixture was stirred for 1 hr at 90° C. The mixture was diluted with EtOAc (100 mL). The organic phase was washed with brine (50 mL*3), dried over anhydrous Na₂SO₄, filtered and concentrated in vacuo. The residue was purified by silica gel chromatography (petroleum ether: EtOAc=50/1-20/1) to afford tert-butyl 2-chloro-4-phenyl-6,7-dihydrothieno[3,2-c]pyridine-5(4H)-carboxylate (1.2 g, 70% yield) as a white solid. LCMS: RT=1.006 min, m/z 294.0 (M-t-butyl+H)⁺.

### 2-chloro-4-phenyl-4,5,6,7-tetrahydrothieno[3,2-c]pyridine Hydrochloride

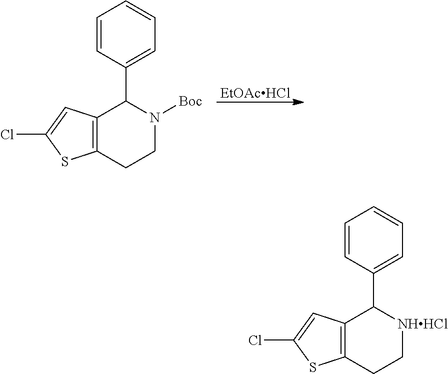

To a solution of tert-butyl 2-chloro-4-phenyl-6,7-dihydrothieno[3,2-c]pyridine-5(4H)-carboxylate (1.2 g, 3.43 mmol) in EtOAc (5 mL) was added 4N HCl in EtOAc (20 mL) dropwise at 20° C. and stirred for 2 h at 20° C. The reaction mixture was concentrated in vacuo to give 2-chloro-4-phenyl-4,5,6,7-tetrahydrothieno[3,2-c]pyridine hydrochloride (700 mg, 2.23 mmol, 65% yield) as a pale yellow solid which was used directly for the next step without further purification. LCMS: RT=0.617 min, m/z 250.0 (M+H)⁺. ¹H NMR (CD₃OD; 400 MHz) δ 7.52-7.50 (m, 3H), 7.41-7.40 (m, 2H), 6.43 (s, 1H), 5.65 (s, 1H), 3.61-3.55 (m, 2H), 3.30-3.17 (m, 2H).

### tert-butyl 2-bromo-4-phenyl-6,7-dihydrothieno[3,2-c]pyridine-5(4H)-carboxylate

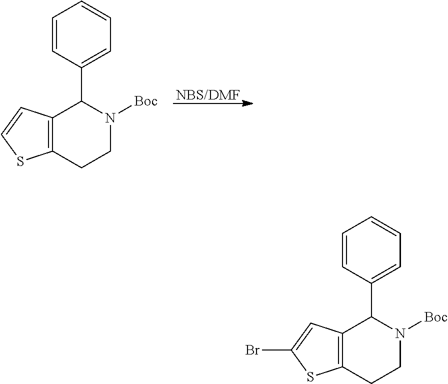

To a solution of tert-butyl 4-phenyl-6,7-dihydrothieno[3,2-c]pyridine-5(4H)-carboxylate (8.30 g, 26.31 mmol) in DMF (40 mL) was added the NBS (5.62 g, 31.58 mmol). Then the mixture was stirred at 30° C. for 1 h. The mixture was diluted with H₂O (200 mL) and extracted with EtOAc (200 mL). The organic layer was dried over Na₂SO₄ and concentrated in vacuo. The residue was purified by column chromatography (petroleum ether: EtOAc=40:1 to 20:1) to give tert-butyl 2-bromo-4-phenyl-6,7-dihydrothieno[3,2-c]pyridine-5(4H)-carboxylate (9.00 g, 22.82 mmol, 87% yield) as a white solid. LCMS: RT=1.078 min, m/z 337.9; 339.9 (M-t-Bu+H)⁺.

### tert-butyl 2-fluoro-4-phenyl-6,7-dihydrothieno[3,2-c]pyridine-5(4H)-carboxylate

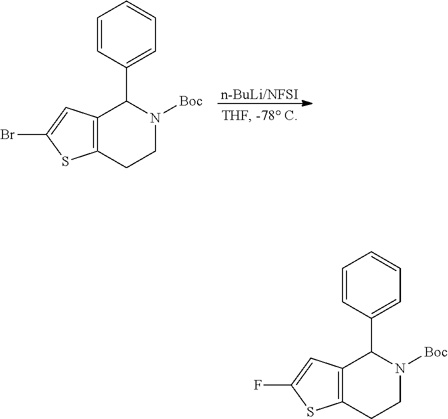

To a solution of tert-butyl 2-bromo-4-phenyl-6,7-dihydrothieno[3,2-c]pyridine-5(4H)-carboxylate (3.0 g, 7.60 mmol) in THF (60 mL) was added n-BuLi (2.5 M, 6.09 mL) at −78° C. under N₂ atmosphere. The mixture was stirred at −78° C. for 30 min. Then a solution of NFSI (5.28 g, 16.74 mmol) in THF (20 mL) was added slowly via syringe. The mixture was stirred at −78° C. for 0.5 h. The mixture was quenched with H₂O (100 mL) and extracted with EtOAc (200 mL). The organic layer was dried over Na₂SO₄ and concentrated in vacuo. The residue was purified by silica gel column chromatography (petroleum ether: EtOAc=100:1 to 40:1) and further purified by prep-HPLC (Column: Phenomenex Synergi Max-RP 250*50 mm*10 um Flow Rate: 80 ml/min, 0.225% formic acid —CH₃CN, 65% CH₃CN to 90% CH₃CN over 35 min) to give tert-butyl 2-fluoro-4-phenyl-6,7-dihydrothieno[3,2-c]pyridine-5(4H)-carboxylate (820.00 mg, 2.41 mmol, 32% yield) as a white solid. LCMS: RT=1.033 min, m/z 278.0 (M-t-Bu+H)⁺. ¹H NMR (CD₃OD, 400 MHz) δ 7.35-7.29 (m, 5H), 6.04 (br, 2 H), 4.31 (m, 0.5 H), 3.12-3.08 (m, 1H), 3.88-2.85 (m, 1.5 H), 2.62-2.59 (m, 1H), 1.50 (s, 9 H)

### 2-fluoro-4-phenyl-4,5,6,7-tetrahydrothieno[3,2-c]pyridine Hydrochloride

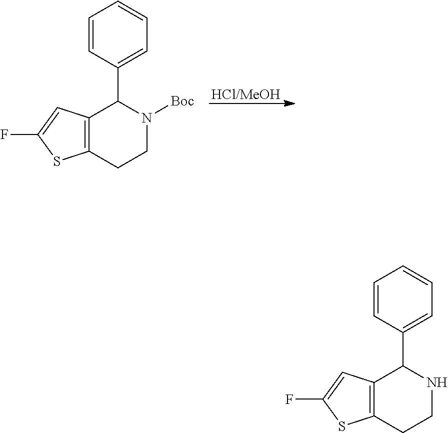

To a solution of tert-butyl 2-fluoro-4-phenyl-6,7-dihydrothieno[3,2-c]pyridine-5(4H)-carboxylate (820 mg, 2.46 mmol) in MeOH (5 mL) was added 4N HCl in MeOH (20 mL). The mixture was stirred at 20° C. for 0.5 h. The mixture was concentrated in vacuo to give 2-fluoro-4-phenyl-4,5,6,7-tetrahydrothieno[3,2-c]pyridine hydrochloride (760 mg) as colorless oil which was used directly for the next steps without further purification. LCMS: RT=0.647 min, m/z 234.1 (M+H)⁺.

### (2-fluoro-4-phenyl-6,7-dihydrothieno[3,2-c]pyridin-5(4H)-yl)(imidazo[1,2-a]pyridin-6-yl)methanone

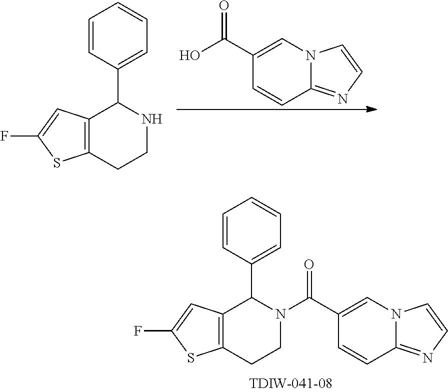 [SMILES: Fc1cc2c(s1)CCNC2c1ccccc1 ; O=C(O)c1ccc2nccn2c1 ; O=C(c1ccc2nccn2c1)N1CCc2sc(F)cc2C1c1ccccc1]

To a solution of 2-fluoro-4-phenyl-4,5,6,7-tetrahydrothieno[3,2-c]pyridine hydrochloride (167 mg, 1.03 mmol) in DMF (8 mL) was added DIPEA (332 mg, 2.57 mmol, 449.16 μL) and imidazo[1,2-a]pyridine-6-carboxylic acid (200 mg, 857.27 μmol) and HATU (489 mg, 1.29 mmol). The mixture was stirred at 20° C. for 2 h. The mixture was concentrated in vacuo and the residue was purified by prep-HPLC (Column: Phenomenex Synergi C18 150*30 mm*4 μm Flow Rate: 25 ml/min, 0.225% formic acid —CH₃CN, 25% CH₃CN to 50% CH₃CN over 20 min) to give (2-fluoro-4-phenyl-6,7-dihydrothieno[3,2-c]pyridin-5(4H)-yl)(imidazo[1,2-a]pyridin-6-yl)methanone (126.00 mg, 333.84 μmol, 39% yield) as a yellow solid. LCMS: RT=2.662 min, m/z 378.0 (M+H)⁺. ¹H NMR (CDCl₃, 400 MHz) δ 8.33 (s, 1 H), 7.72 (s, 1H), 7.71 (d, J=2.0 Hz, 1H), 7.63 (s, 1H), 7.36-7.27 (m, 6H), 7.17 (d, J=2.0 Hz, 1H), 6.13 (s, 1H), 4.02-3.94 (m, 1H), 3.61-3.37 (m, 1H), 3.02-2.96 (m, 1H), 2.70-2.68 (m, 1H).

### Example 4

### 3-fluoro-N-(2-(thiophen-2-yl)ethyl)benzamide

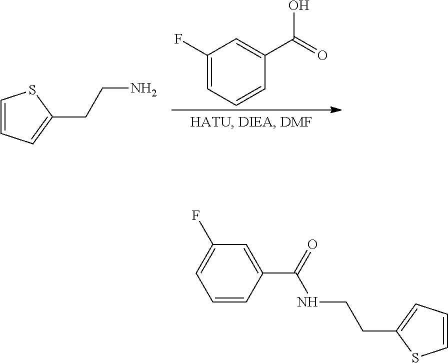 [SMILES: NCCc1cccs1 ; O=C(O)c1cccc(F)c1 ; O=C(NCCc1cccs1)c1cccc(F)c1]

To a solution of 3-fluorobenzoic acid (500 mg, 3.57 mmol, 1.00 eq) and HATU (2.0 g, 5.35 mmol) in DMF (10 mL) at 0° C. was added DIPEA (1.4 g, 10.71 mmol) dropwise. The mixture was stirred at 0° C. for 30 min. Then 2-(thiophen-2-yl)ethan-1-amine (499.36 mg, 3.93 mmol) was added and stirred for 2 h at 20° C. The mixture was diluted with CH₂Cl₂ (50 mL) and washed with sat.NaHCO₃ aq (30 mL*2). The organic layer was separated and washed by 1N HCl (30 mL*2) and brine (30 mL*2), dried over Na₂SO₄ and filtered, concentrated in vacuo to give 3-fluoro-N-(2-(thiophen-2-yl)ethyl)benzamide (1.05 g, crude) as a brown solid. The residue was used directly for the next step without further purification. LCMS: RT=0.732 min, m/z 250.1 (M+H)⁺

### 4-(3-fluorophenyl)-6,7-dihydrothieno[3,2-c]pyridine

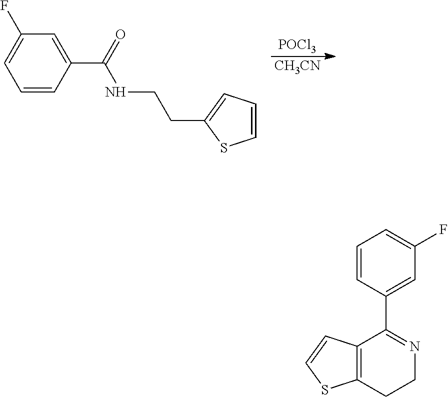

To a solution of 3-fluoro-N-(2-(thiophen-2-yl)ethyl)benzamide (1.05 g, 4.21 mmol) in CH₃CN (10.00 mL) was added POCl₃ (3.23 g, 21.05 mmol) in one portion. The mixture was stirred at 80° C. for 3 h. The mixture was concentrated in vacuo and the residue was diluted with CH₂Cl₂ (50 mL) and quenched with sat.NaHCO₃ aq (30 mL*2). The organic layer was washed with brine (30 mL), dried over Na₂SO₄ and filtered, concentrated in vacuo to give 4-(3-fluorophenyl)-6,7-dihydrothieno[3,2-c]pyridine (910.00 mg, crude) as a brown oil. The residue was used directly for next step without further purification. LCMS: RT=0.476 min, m/z 232.0 (M+H)⁺.

### 4-(3-fluorophenyl)-4,5,6,7-tetrahydrothieno[3,2-c]pyridine

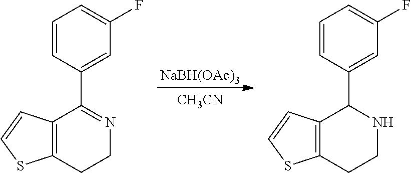

To a solution of 4-(3-fluorophenyl)-6,7-dihydrothieno[3,2-c]pyridine (909 mg, 3.93 mmol) in CH₃CN (10 mL) at 20° C. was added NaBH(OAc)₃ (1.67 g, 7.86 mmol) in portion wise. The mixture was stirred at 20° C. for 5 h. The mixture was concentrated in vacuo and the residue was diluted with H₂O (50 mL). The aqueous phase was acidified with 1N HCl till pH=3. The mixture was extracted with EtOAc (50 mL*2) and the aqueous phase was basified with aqueous NaHCO₃ till pH=8 and extracted with EtOAc (50 mL*2). The combined organic layers were washed with brine (50 mL*2), dried with anhydrous Na₂SO₄, filtered and concentrated in vacuo to give 4-(3-fluorophenyl)-4,5,6,7-tetrahydrothieno[3,2-c]pyridine (600 mg, 2.49 mmol, 63.5% yield) as a pale yellow solid. LCMS: RT=0.549 min, m/z 234.2 (M+H)⁺.

### tert-butyl 4-(3-fluorophenyl)-6,7-dihydrothieno[3,2-c]pyridine-5(4H)-carboxylate

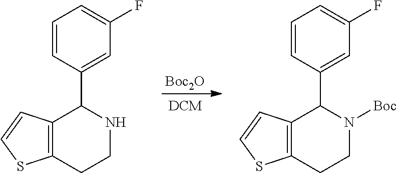 [SMILES: "Boc2O" ; Fc1cccc(C2NCCc3sccc32)c1 ; CC(C)(C)OC(=O)N1CCc2sccc2C1c1cccc(F)c1]

To a solution of 4-(3-fluorophenyl)-4,5,6,7-tetrahydrothieno[3,2-c]pyridine (600 mg, 2.57 mmol) in CH₂Cl₂ (10 mL) was added Boc₂O (842 mg, 3.86 mmol) and NEt₃ (781 mg, 7.72 mmol) at 20° C. The mixture was stirred for 2 h at 20° C. The solvent was removed in vacuo and the residue was dissolved in EtOAc (50 mL) and 1 g of N,N-dimethylethylenediamine was added to remove the excess Boc₂O. The mixture was stirred for 30 min then the organic phase was washed with 1N HCl (20 mL*4), brine (20 mL*2), dried with anhydrous Na₂SO₄, filtered and concentrated in vacuo to give tert-butyl 4-(3-fluorophenyl)-6,7-dihydrothieno[3,2-c]pyridine-5(4H)-carboxylate (860 mg, crude) as yellow oil which was used directly for next step without further purification. LCMS: RT=0.955 min, m/z 278.1 (M-t-Butyl+H)⁺

### tert-butyl 2-chloro-4-(3-fluorophenyl)-6,7-dihydrothieno[3,2-c]pyridine-5(4H)-carboxylate

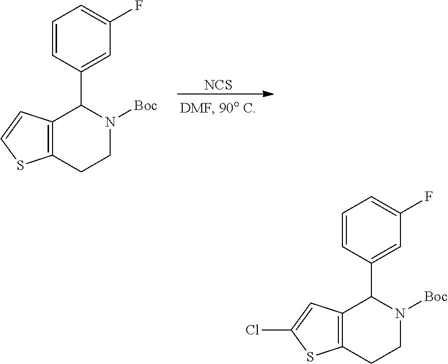 [SMILES: CC(C)(C)OC(=O)N1CCc2sccc2C1c1cccc(F)c1 ; "NCS" ; CC(C)(C)OC(=O)N1CCc2sc(Cl)cc2C1c1cccc(F)c1]

To a solution of tert-butyl 4-(3-fluorophenyl)-6,7-dihydrothieno[3,2-c]pyridine-5(4H)-carboxylate (860 mg, 2.58 mmol) in DMF (10 mL) was added NCS (379 mg, 2.84 mmol). The reaction mixture was stirred for 1 h at 90° C. The reaction mixture was added EtOAc (50 mL). The organic phase was washed with brine (30 mL*3), dried with anhydrous Na₂SO₄, filtered and concentrated in vacuo. The residue was purified by silica gel chromatography (PE/EtOAc=50/1, 20/1) to give tert-butyl 2-chloro-4-(3-fluorophenyl)-6,7-dihydrothieno[3,2-c]pyridine-5(4H)-carboxylate (600 mg, 1.44 mmol, 55.6% yield) as a yellow oil.

### 2-chloro-4-(3-fluorophenyl)-4,5,6,7-tetrahydrothieno[3,2-c]pyridine Hydrochloride

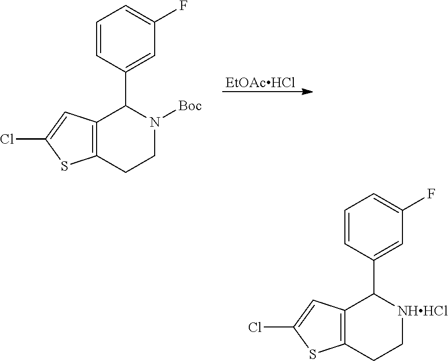

To a solution of tert-butyl 2-chloro-4-(3-fluorophenyl)-6,7-dihydrothieno[3,2-c]pyridine-5(4H)-carboxylate (600 mg, 1.63 mmol) in EtOAc (2 mL) was added 4N HCl in EtOAc (10 mL) at 20° C. and stirred for 2 h at 20° C. The mixture was concentrated in vacuo to give 2-chloro-4-(3-fluorophenyl)-4,5,6,7-tetrahydrothieno[3,2-c]pyridine hydrochloride (500 mg, 1.10 mmol, 67.6% yield) as a yellow solid. LCMS: RT=0.619 min, m/z 268.0 (M+H)⁺. ¹H NMR (CD₃OD; 400 MHz) δ 7.55-7.51 (m, 1H), 7.28-7.23 (m, 2H), 7.19-7.17 (m, 1H), 6.47 (s, 1H), 5.70 (s, 1H), 3.60-3.57 (m, 2H), 3.18-3.17 (m, 2H).

### (2-chloro-4-(3-fluorophenyl)-6,7-dihydrothieno[3,2-c]pyridin-5(4H)-yl)(imidazo[1,2-a]pyridin-6-yl)methanone

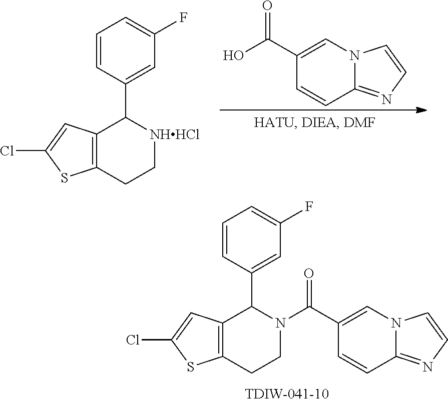

To a solution of 2-chloro-4-(3-fluorophenyl)-4,5,6,7-tetrahydrothieno[3,2-c]pyridine hydrochloride (80 mg, 493.37 μmol) in DMF (10 mL) was added HATU (281 mg, 740.06 mol) and DIPEA (319 mg, 2.47 mmol) at 0° C. The mixture was stirred for 0.5 h and then imidazo[1,2-a]pyridine-6-carboxylic acid (246 mg, 542.71 μmol) was added. The mixture was stirred for 2 h at 20° C. The mixture was diluted with H₂O (10 mL) and EtOAc (20 mL). The mixture was extracted with EtOAc (10 mL*2). The combined organic phase was washed with brine (20 mL*3), dried with anhydrous Na₂SO₄, filtered and concentrated in vacuo. The residue was purified by prep-HPLC (Phenomenex Synergi C18 150*30 mm*4 um, Flow Rate: 28 (ml/min), 0.05% HCl—CH₃CN, 35% CH₃CN to 55% CH₃CN over 9 min) to give (2-chloro-4-(3-fluorophenyl)-6,7-dihydrothieno[3,2-c]pyridin-5(4H)-yl)(imidazo[1,2-a]pyridin-6-yl)methanone (46.00 mg, 111.08 μmol, 22.5% yield) as a pale yellow solid. LCMS: RT=2.639 min, m/z 412.1 (M+H)⁺. ¹H NMR (CD3OD, 400 MHz) δ 9.01 (s, 1H), 8.27 (d, J=2.0 Hz, 1H), 8.12 (d, J=2.0 Hz, 1H), 8.03-7.96 (m, 2H), 7.42-7.40 (m, 1H), 7.18-7.10 (m, 3H), 6.79 (br, 1H), 6.68 (s, 1H), 3.92 (brs, 1H), 3.53-3.49 (m, 1H), 3.10-3.06 (m, 1H), 2.89-2.85 (m, 1H).

### Example 5

### (2-chloro-4-(4-fluorophenyl)-6,7-dihydrothieno[3,2-c]pyridin-5(4H)-yl)(imidazo[1,2-a]pyridin-6-yl)methanone

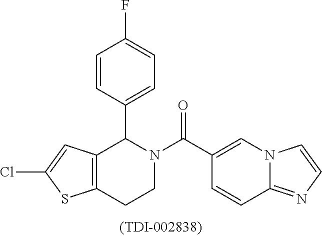 [SMILES: O=C(c1ccc2nccn2c1)N1CCc2sc(Cl)cc2C1c1ccc(F)cc1]

According to the procedure of Example 4, (2-chloro-4-(4-fluorophenyl)-6,7-dihydrothieno[3,2-c]pyridin-5(4H)-yl)(imidazo[1,2-a]pyridin-6-yl)methanone was prepared. LCMS: RT=2.456 min, m/z 412.0 (M+H)⁺. 1H NMR: (DMSO-d6; 400 MHz) δ 9.11 (s, 1H), 8.31 (s, 1H), 8.30 (s, 1H), 8.02-8.00 (d, J=8.0 Hz, 1H), 7.93 (m, 1H), 7.39-7.21 (m, 4 H), 6.91 (brs, 1H), 6.72 (brs, 1H), 3.84 (m, 1H), 3.09-3.03 (m, 2H), 2.85-2.81 (m, 1H).

### Example 6

### 4-(2-fluorophenyl)-4,5,6,7-tetrahydrothieno[3,2-c]pyridine

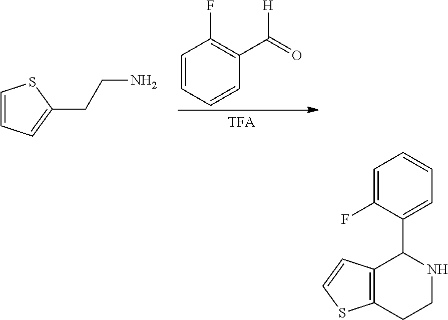 [SMILES: NCCc1cccs1 ; O=Cc1ccccc1F ; Fc1ccccc1C1NCCc2sccc21]

A mixture of 2-(thiophen-2-yl)ethan-1-amine (1.00 g, 7.86 mmol) and 2-fluorobenzaldehyde (975 mg, 7.86 mmol) in TFA (20.00 mL) was heated to 60° C. for 2 h. The reaction mixture was poured into sat.Na₂CO₃ aq (50 mL). The mixture was extracted with EtOAc (60 mL). The organic layer was dried over Na₂SO₄, filtered and concentrated in vacuo. The residue was purified by column chromatography (PE to PE/EtOAc=4/1) to give 4-(2-fluorophenyl)-4,5,6,7-tetrahydrothieno[3,2-c]pyridine as colorless gum. LCMS: RT=0.581 min, m/z 234.1 [M+H]⁺. ¹H NMR (400 MHz, CDCl3) δ 7.27-7.25 (m, 1H), 7.09-7.04 (m, 4H), 6.52 (d, J=5.2 Hz, 1H), 5.44 (s, 1H), 3.27-3.23 (m, 1H), 3.14-3.13 (m, 1H), 2.95-2.89 (m, 2H).

### (2-chloro-4-(2-fluorophenyl)-6,7-dihydrothieno[3,2-c]pyridin-5(4H)-yl)(imidazo[1,2-a]pyridin-6-yl)methanone

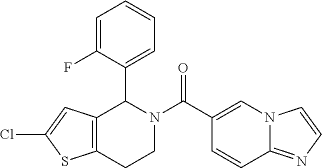 [SMILES: O=C(c1ccc2nccn2c1)N1CCc2sc(Cl)cc2C1c1ccccc1F]

According to the procedure of Example 4, (2-chloro-4-(2-fluorophenyl)-6,7-dihydrothieno[3,2-c]pyridin-5(4H)-yl)(imidazo[1,2-a]pyridin-6-yl)methanone was prepared. LCMS: RT=2.628 min; m/z 412.0 (M+H)⁺. ¹H NMR: (CDCl₃, 400 MHz) δ 8.31 (s, 1H), 7.70 (s, 1H), 7.64 (s, 1H), 7.62 (s, 1H), 7.33-7.31 (m, 1H), 7.14-7.09 (m, 4H), 6.85-6.60 (m, 1H), 6.50 (s, 1H), 4.33-4.23 (m, 1H), 3.49-3.45 (m, 1H), 2.93-2.82 (m, 1H), 2.81-2.77 (m, 1H).

### Example 7

### tert-butyl 2-bromo-4-(2-fluorophenyl)-6,7-dihydrothieno[3,2-c]pyridine-5(4H)-carboxylate

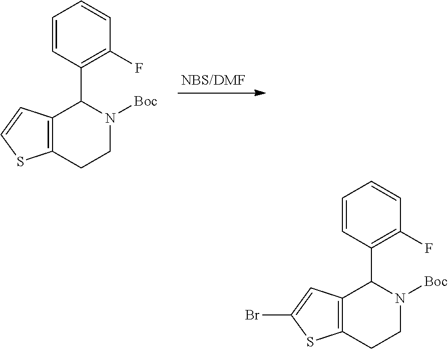 [SMILES: CC(C)(C)OC(=O)N1CCc2sccc2C1c1ccccc1F ; CC(C)(C)OC(=O)N1CCc2sc(Br)cc2C1c1ccccc1F]

To a solution of tert-butyl 4-(2-fluorophenyl)-6,7-dihydrothieno[3,2-c]pyridine-5(4H)-carboxylate (4.10 g, 12.30 mmol) in DMF (40 mL) was added the NBS (2.19 g, 12.30 mmol). Then the mixture was stirred at 30° C. for 1 h. The mixture was diluted with water (200 mL) and extracted with EtOAc (200 mL). The organic layer was dried over Na₂SO₄ and concentrated in vacuo. The residue was purified by column chromatograph (PE: EtOAc=40:1 to 20:1) to give tert-butyl 2-bromo-4-(2-fluorophenyl)-6,7-dihydrothieno[3,2-c]pyridine-5(4H)-carboxylate (4.20 g, 8.66 mmol, 70.4% yield) as yellow oil. LCMS: RT=1.080 min, m/z 355.9; 357.9 (M-t-Bu+H)⁺.

### tert-butyl 2-fluoro-4-(2-fluorophenyl)-6,7-dihydrothieno[3,2-c]pyridine-5(4H)-carboxylate

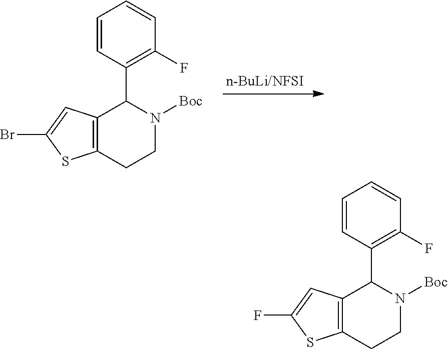 [SMILES: CC(C)(C)OC(=O)N1CCc2sc(Br)cc2C1c1ccccc1F ; CC(C)(C)OC(=O)N1CCc2sc(F)cc2C1c1ccccc1F]

To a solution of tert-butyl 2-bromo-4-(2-fluorophenyl)-6,7-dihydrothieno[3,2-c]pyridine-5(4H)-carboxylate (2.00 g, 4.85 mmol) in THF (60 mL) at −78° C. was added n-BuLi (2.5 M, 3.88 mL) under N₂ atmosphere. Then the mixture was stirred at −78° C. for 30 min. Then a solution of NFSI (3.36 g, 10.67 mmol) in THF (10 mL) was added slowly via syringe. Then the mixture was stirred at −78° C. for 0.5 h. The reaction was quenched with water (100 mL) and extracted with EtOAc (200 mL). The organic layer was dried over Na₂SO₄ and concentrated in vacuo. The residue was purified by silica gel column chromatograph (PE:EtOAc=100:1 to 40:1) to give tert-butyl 2-fluoro-4-(2-fluorophenyl)-6,7-dihydrothieno[3,2-c]pyridine-5(4H)-carboxylate (800 mg, crude) as colorless oil. LCMS: RT=1.073 min, m/z 296.1 (M-t-Bu+H)⁺.

### 2-fluoro-4-(2-fluorophenyl)-4,5,6,7-tetrahydrothieno[3,2-c]pyridine

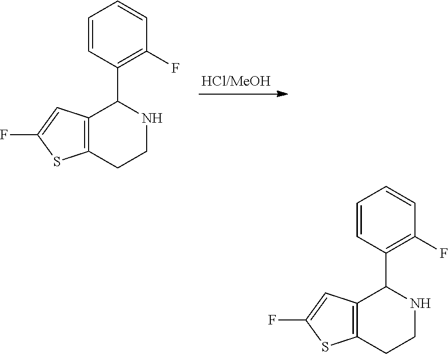 [SMILES: Fc1cc2c(s1)CCNC2c1ccccc1F ; Fc1cc2c(s1)CCNC2c1ccccc1F]

To a solution of tert-butyl 2-fluoro-4-(2-fluorophenyl)-6,7-dihydrothieno[3,2-c]pyridine-5(4H)-carboxylate (800 mg, 2.28 mmol) in MeOH (5 ml) was added the HCl/MeOH (4 mol/L, 20 mL). Then the mixture was stirred at 20° C. for 0.5 h. The mixture was concentrated in vacuo and the residue was purified by prep-HPLC (Column: Phenomenex Synergi C18 150*30 mm*4 um Flow Rate: 25 ml/min, 0.225% formic acid-CH₃CN, 4% CH₃CN to 34% CH₃CN over 12 min) to give 2-fluoro-4-(2-fluorophenyl)-4,5,6,7-tetrahydrothieno[3,2-c]pyridine (130.00 mg, 496.64 μmol, 21.78% yield) as colorless oil. LCMS: RT=0.606 min, m/z 252.2 (M+H)⁺. ¹H NMR (CDCl₃, 400 MHz) δ 7.28-7.27 (m, 1H), 7.26-7.16 (m, 1H), 7.14-7.06 (m, 2H), 5.88 (s, 1H), 5.28 (s, 1H), 3.27-3.22 (m, 1H), 3.16-3.13 (m, 1H), 3.13-2.71 (m, 2H).

### (2-fluoro-4-(2-fluorophenyl)-6,7-dihydrothieno[3,2-c]pyridin-5(4H)-yl)(imidazo[1,2-a]pyridin-6-yl)methanone

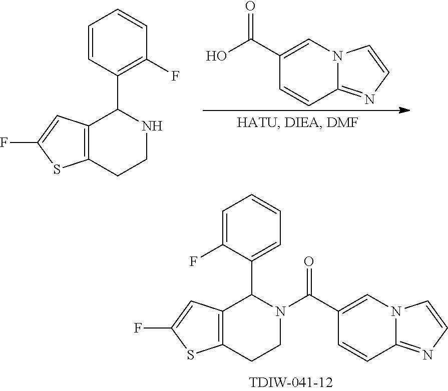 [SMILES: Fc1cc2c(s1)CCNC2c1ccccc1F ; O=C(O)c1ccc2nccn2c1 ; O=C(c1ccc2nccn2c1)N1CCc2sc(F)cc2C1c1ccccc1F]

To a solution of the imidazo[1,2-a]pyridine-6-carboxylic acid (109 mg, 672.53 mol) in DMF (8 mL) was added the DIPEA (201 mg, 1.55 mmol, 271.05 μL) and HATU (295 mg, 776.00 μmol). Then 2-fluoro-4-(2-fluorophenyl)-4,5,6,7-tetrahydrothieno[3,2-c]pyridine (130 mg, 517.33 μmol) was added and stirred at 20° C. for 2 h. The mixture was concentrated in vacuo and the residue was purified by prep-HPLC (Column: Phenomenex Synergi C18 150*30 mm*4 um; Flow Rate: 25 ml/min, 0.225% formic acid-CH₃CN, 25% CH₃CN to 55% CH₃CN over 25 min) to give (2-fluoro-4-(2-fluorophenyl)-6,7-dihydrothieno[3,2-c]pyridin-5(4H)-yl)(imidazo[1,2-a]pyridin-6-yl)methanone (65.00 mg, 160.44 μmol, 31.01% yield) as a yellow solid. LCMS: RT=2.543 min, m/z 396.1 (M+H)⁺. ¹H NMR (CDCl₃; 400 MHz) δ 8.24 (s, 1H), 7.63 (s, 1H), 7.57-7.54 (m, 2H), 7.33-7.31 (m, 1H), 7.07-7.05 (m, 4H), 6.61-6.44 (m, 1H), 6.01 (s, 1H), 4.39-4.15 (m, 1H), 3.57-3.32 (m, 1 H), 2.94-2.65 (m, 2H).

### Example 8

### (2-fluoro-4-(3-fluorophenyl)-6,7-dihydrothieno[3,2-c]pyridin-5(4H)-yl)(imidazo[1,2-a]pyridin-6-yl)methanone

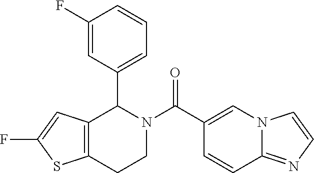 [SMILES: O=C(c1ccc2nccn2c1)N1CCc2sc(F)cc2C1c1cccc(F)c1]

According to the procedure of Example 6, (2-fluoro-4-(3-fluorophenyl)-6,7-dihydrothieno[3,2-c]pyridin-5(4H)-yl)(imidazo[1,2-a]pyridin-6-yl)methanone was prepared. LCMS: RT=2.587 min, m/z 396.1 (M+H)⁺. 1H NMR: (CDCl₃; 400 MHz) δ 8.27 (s, 1H), 7.64 (s, 1H), 7.59-7.56 (m, 2H), 7.26-7.24 (m, 1H), 7.09-6.92 (m, 4H), 6.62 (br, 1H), 6.05 (s, 1H), 3.94-3.84 (m, 1H), 3.38-3.32 (m, 1H), 2.91-2.88 (m, 1H), 2.66-2.62 (m, 1H).

### Example 9

### (2-fluoro-4-(4-fluorophenyl)-6,7-dihydrothieno[3,2-c]pyridin-5(4H)-yl)(imidazo[1,2-a]pyridin-6-yl)methanone

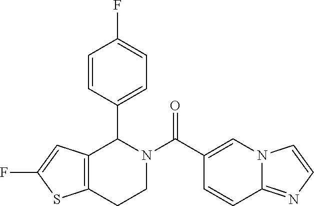 [SMILES: O=C(c1ccc2nccn2c1)N1CCc2sc(F)cc2C1c1ccc(F)cc1]

According to the procedure of Example 6, (2-fluoro-4-(4-fluorophenyl)-6,7-dihydrothieno[3,2-c]pyridin-5(4H)-yl)(imidazo[1,2-a]pyridin-6-yl)methanone was prepared. LCMS: RT=2.575 min, m/z 396.1 (M+H)⁺. ¹H NMR (CDCl₃; 400 MHz) δ 8.34 (s, 1H), 7.73 (s, 1H), 7.68-7.66 (m, 2H), 7.27-7.25 (m, 2H), 7.16 (d, J=1.6 Hz, 1H), 7.06 (t, J=8.4 Hz, 2H), 6.71 (br, 1H), 6.12 (s, 1H), 3.99-3.95 (m, 1H), 3.43-3.40 (m, 1H), 3.01-2.95 (m, 1H), 2.76-2.70 (m, 1H).

### Example 10

### (S)-4-phenyl-4,5,6,7-tetrahydrothieno[3,2-c]pyridine and (R)-4-phenyl-4,5,6,7-tetrahydrothieno[3,2-c]pyridine

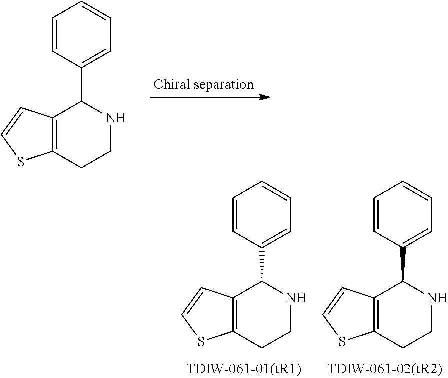

rac-4-phenyl-4,5,6,7-tetrahydrothieno[3,2-c]pyridine (7.10 g, 32.98 mmol) was separated by SFC (column:OD (250 mm*30 mm, 10 um, Condition:Base-MeOH, Mobile phase: A for CO₂ and B for Methanol (0.1% Ammonia), Gradient: B 30%, Flow rate: 60 mL/min, Back pressure: 100 bar, column temperature: 35° C., Gradient Time (min): 2.5 minutes) to give (S)-4-phenyl-4,5,6,7-tetrahydrothieno[3,2-c]pyridine (tR1, 3.40 g, 15.61 mmol, 47% yield, 98.73% ee) as a yellow solid and (R)-4-phenyl-4,5,6,7-tetrahydrothieno[3,2-c]pyridine (tR2, 3.40 g, 15.62 mmol, 47% yield, 97.32% ee) as a yellow solid. LCMS of (S)-4-phenyl-4,5,6,7-tetrahydrothieno[3,2-c]pyridine (tR1): RT=2.571 min, m/z 216.1 [M+H]⁺. LCMS of (R)-4-phenyl-4,5,6,7-tetrahydrothieno[3,2-c]pyridine (tR2): RT=2.558 min, m/z 216.1 [M+H]⁺. 1H NMR of (S)-4-phenyl-4,5,6,7-tetrahydrothieno[3,2-c]pyridine (tR1): (CDCl₃, 400 MHz) δ 7.35-7.28 (m, 5H), 7.01 (d, J=5.2 Hz, 1H), 6.47 (d, J=5.2 Hz, 1H), 5.04 (s, 1H), 3.35-3.30 (m, 1H), 3.14-3.13 (m, 1H), 3.09-2.98 (m, 1H), 2.98-2.88 (m, 1H). ¹H NMR of (R)-4-phenyl-4,5,6,7-tetrahydrothieno[3,2-c]pyridine (tR2): (CDCl₃, 400 MHz) δ 7.35-7.28 (m, 5H), 7.02 (d, J=5.2 Hz, 1H), 6.48 (d, J=5.2 Hz, 1H), 5.04 (s, 1H), 3.35-3.30 (m, 1H), 3.14-3.12 (m, 1H), 3.09-2.98 (m, 1H), 2.98-2.88 (m, 1H).

### (S)-1-methyl-5-(4-phenyl-4,5,6,7-tetrahydrothieno[3,2-c]pyridine-5-carbonyl)pyridin-2(1H)-one

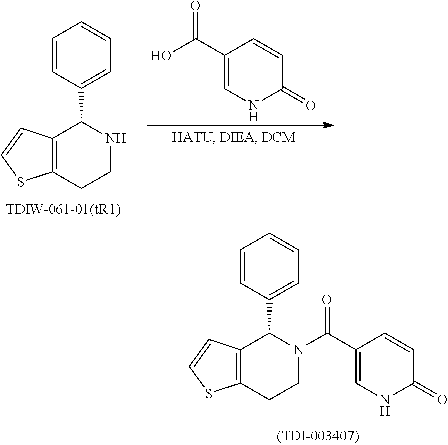 [SMILES: c1ccc([C@@H]2NCCc3sccc32)cc1 ; O=C(O)c1ccc(=O)[nH]c1 ; O=C(c1ccc(=O)[nH]c1)N1CCc2sccc2[C@@H]1c1ccccc1]

To a mixture of 1-methyl-6-oxo-1,6-dihydropyridine-3-carboxylic acid (2.27 g, 16.35 mmol) and HATU (8.48 g, 22.29 mmol) in CH₂Cl₂ (40 mL) were added (S)-4-phenyl-4,5,6,7-tetrahydrothieno[3,2-c]pyridine (3.20 g, 14.86 mmol) and DIPEA (3.84 g, 29.72 mmol, 5.19 mL) in one portion. The mixture was stirred at 25° C. for 12 h. The mixture was quenched with sat.Na₂CO₃ aq (300 mL) and extracted with CH₂Cl₂ (200 mL*3). The combined organic phase was washed with brine (200 mL), dried with anhydrous Na₂SO₄, filtered and concentrated in vacuo. The crude product was purified by prep-HPLC (Phenomenex Gemini C18 250*50 10u, Flow Rate: 80 (ml/min), water (0.05% ammonia hydroxide v/v) —CH₃CN, 30% CH₃CN to 49% CH₃CN over 32 min) to give (S)-1-methyl-5-(4-phenyl-4,5,6,7-tetrahydrothieno[3,2-c]pyridine-5-carbonyl)pyridin-2(1H)-one (2.91 g, 8.47 mmol, 57% yield, 95.43% ee) as a white solid. LCMS: RT=2.078 min, m/z 337.0 [M+H]⁺. ¹H NMR: (CDCl₃, 400 MHz) δ 12.93 (br, 1H), 7.60-7.55 (m, 2H), 7.32-7.30 (m, 5 H), 7.20 (d, J=5.2 Hz, 1H), 6.75 (d, J=5.2 Hz, 2H), 6.59 (d, J=9.6 Hz, 1H), 4.08 (s, 1 H), 3.44-3.41 (m, 1H), 3.05-2.94 (m, 1H), 2.91-2.90 (m, 1H).

### (R)-1-methyl-5-(4-phenyl-4,5,6,7-tetrahydrothieno[3,2-c]pyridine-5-carbonyl)pyridin-2(1H)-one

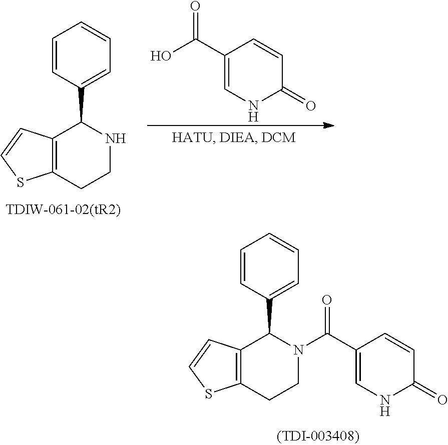 [SMILES: c1ccc([C@H]2NCCc3sccc32)cc1 ; O=C(O)c1ccc(=O)[nH]c1 ; O=C(c1ccc(=O)[nH]c1)N1CCc2sccc2[C@H]1c1ccccc1]

To a mixture of 1-methyl-6-oxo-1,6-dihydropyridine-3-carboxylic acid (2.27 g, 16.35 mmol) and HATU (8.48 g, 22.29 mmol) in CH₂Cl₂ (40 mL) were added (R)-4-phenyl-4,5,6,7-tetrahydrothieno[3,2-c]pyridine (3.20 g, 14.86 mmol) and DIPEA (3.84 g, 29.72 mmol, 5.19 mL) in one portion. Then the mixture was stirred at 25° C. for 12 h. The mixture was quenched with sat.Na₂CO₃ aq (300 mL) and extracted with CH₂Cl₂ (200 mL*3). The combined organic phase was washed with brine (200 mL), dried with anhydrous Na₂SO₄, filtered and concentrated in vacuo. The crude product was purified by prep-HPLC (Phenomenex Gemini C18 250*50 mm*10 um, Flow Rate: 80 (ml/min), water (0.05% ammonia hydroxide v/v) —CH₃CN, 30% CH₃CN to 50% CH₃CN over 30 min) to give (R)-1-methyl-5-(4-phenyl-4,5,6,7-tetrahydrothieno[3,2-c]pyridine-5-carbonyl)pyridin-2(1H)-one (3.74 g, 11.10 mmol, 75% yield, 97.71% ee) as a white solid. LCMS: RT=2.081 min, m/z 337.0 [M+H]⁺. ¹H NMR: (CDCl₃, 400 MHz) δ 12.81 (br, 1H), 7.62-7.56 (m, 2H), 7.35-7.31 (m, 5H), 7.21 (d, J=5.2 Hz, 1H), 6.76 (d, J=4.0 Hz, 2H), 6.60 (d, J=9.2 Hz, 1H), 4.09 (s, 1H), 3.45-3.42 (m, 1H), 3.07-2.95 (m, 1H), 2.92-2.91 (m, 1H).

### Example 11

### (S)-2-chloro-4-(4-fluorophenyl)-4,5,6,7-tetrahydrothieno[3,2-c]pyridine and (R)-2-chloro-4-(4-fluorophenyl)-4,5,6,7-tetrahydrothieno[3,2-c]pyridine

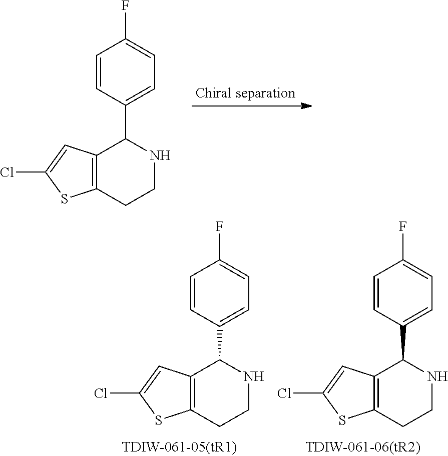

rac-2-chloro-4-(4-fluorophenyl)-4,5,6,7-tetrahydrothieno[3,2-c]pyridine (7.00 g, 26.14 mmol, 1.00 eq) was separated by SFC (Column:OD (250 mm*30 mm, 10 um, Condition:Base-MeOH, Mobile phase: A for CO₂ and B for Methanol (0.1% Ammonia), Gradient: B 30%, Flow rate: 60 mL/min, Back pressure: 100 bar, Column temperature: 35° C., Gradient Time (min): 2.5 minutes) to give (S)-2-chloro-4-(4-fluorophenyl)-4,5,6,7-tetrahydrothieno[3,2-c]pyridine (tR1, 2.80 g, 10.25 mmol, 39% yield, >99% ee) as a white solid and (R)-2-chloro-4-(4-fluorophenyl)-4,5,6,7-tetrahydrothieno[3,2-c]pyridine (3.00 g, 11.09 mmol, 42% yield, 98.52% ee) as a white solid. LCMS for (S)-2-chloro-4-(4-fluorophenyl)-4,5,6,7-tetrahydrothieno[3,2-c]pyridine: RT=2.964 min, m/z 268.0 [M+H]⁺. LCMS for (R)-2-chloro-4-(4-fluorophenyl)-4,5,6,7-tetrahydrothieno[3,2-c]pyridine: RT=2.960 min, m/z 268.0 [M+H]⁺. 1H NMR for (S)-2-chloro-4-(4-fluorophenyl)-4,5,6,7-tetrahydrothieno[3,2-c]pyridine: (CDCl₃, 400 MHz) δ 7.27-7.25 (m, 2H), 7.04 (t, J=8.8 Hz, 2H), 6.26 (s, 1H), 4.91 (s, 1H), 3.33-3.28 (m, 1H), 3.16-3.12 (m, 1H), 2.91-2.88 (m, 1H), 2.88-2.75 (m, 1H). ¹H NMR for (R)-2-chloro-4-(4-fluorophenyl)-4,5,6,7-tetrahydrothieno[3,2-c]pyridine: (CDCl₃, 400 MHz) δ 7.27-7.25 (m, 5H), 7.04 (t, J=8.8 Hz, 2H), 6.26 (s, 1H), 4.91 (s, 1H), 3.33-3.28 (m, 1H), 3.15-3.12 (m, 1H), 2.91-2.88 (m, 1H), 2.88-2.75 (m, 1H).

### (S)-(2-chloro-4-(4-fluorophenyl)-6,7-dihydrothieno[3,2-c]pyridin-5(4H)-yl)(imidazo[1,2-a]pyridin-6-yl)methanone

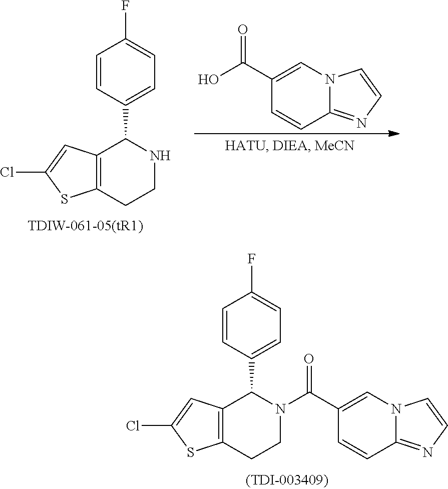 [SMILES: Fc1ccc([C@@H]2NCCc3sc(Cl)cc32)cc1 ; O=C(O)c1ccc2nccn2c1 ; O=C(c1ccc2nccn2c1)N1CCc2sc(Cl)cc2[C@@H]1c1ccc(F)cc1]

To a solution of imidazo[1,2-a]pyridine-6-carboxylic acid (1.87 g, 11.51 mmol) in CH₃CN (20 mL) were added HATU (7.95 g, 20.92 mmol) and DIPEA (4.06 g, 31.38 mmol, 5.48 mL) and (S)-2-chloro-4-(4-fluorophenyl)-4,5,6,7-tetrahydrothieno[3,2-c]pyridine (2.80 g, 10.46 mmol). The mixture was stirred at 25° C. for 1 h. The reaction mixture was concentrated in vacuo to remove solvent. The residue was diluted with water (100 mL) and extracted with EtOAc 300 mL (100 mL*3). The combined organic layers were dried over Na₂SO₄, filtered and concentrated in vacuo to give a residue. The residue was purified by prep-HPLC (Phenomenex Gemini C18 250*50 mm*10 um, Flow Rate: 80 (ml/min), water (0.05% ammonia hydroxide v/v) —CH₃CN, 25% CH₃CN to 55% CH₃CN over 30 min) to give (S)-(2-chloro-4-(4-fluorophenyl)-6,7-dihydrothieno[3,2-c]pyridin-5(4H)-yl)(imidazo[1,2-a]pyridin-6-yl)methanone (3.12 g, 7.58 mmol, 72% yield, 99.17% ee) as a gray solid. LCMS: RT=2.874 min, m/z 412.1 [M+H]⁺. ¹H NMR: (CDCl₃, 400 MHz) δ 8.33 (s, 1H), 7.72 (s, 1 H), 7.67-7.63 (m, 2H), 7.34 (s, 2H), 7.13 (d, J=8.0 Hz, 1H), 7.05 (t, J=8.4 Hz, 2H), 6.77 (br, 1H), 6.56 (s, 1H), 3.95 (br, 1H), 3.40-3.39 (m, 1H), 3.02-2.95 (m, 1H), 2.82-2.77 (m, 1 H).

### (R)-(2-chloro-4-(4-fluorophenyl)-6,7-dihydrothieno[3,2-c]pyridin-5(4H)-yl)(imidazo[1,2-a]pyridin-6-yl)methanone

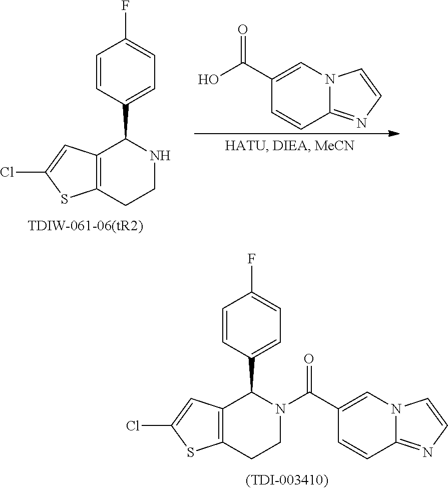 [SMILES: Fc1ccc([C@H]2NCCc3sc(Cl)cc32)cc1 ; O=C(O)c1ccc2nccn2c1 ; O=C(c1ccc2nccn2c1)N1CCc2sc(Cl)cc2[C@H]1c1ccc(F)cc1]

To a solution of imidazo[1,2-a]pyridine-6-carboxylic acid (2.13 g, 13.15 mmol) in CH₃CN (20 mL) was added HATU (9.09 g, 23.90 mmol) and DIPEA (4.63 g, 35.85 mmol, 6.26 mL) and (R)-2-chloro-4-(4-fluorophenyl)-4,5,6,7-tetrahydrothieno[3,2-c]pyridine (3.20 g, 11.95 mmol). The mixture was stirred at 25° C. for 1 h. The solvent was removed in vacuo. The residue was diluted with H₂O (100 mL) and extracted with EtOAc (100 mL*3). The combined organic layers were dried over Na₂SO₄, filtered and concentrated in vacuo to give a residue. The residue was purified by prep-HPLC (Phenomenex Gemini C18 250*50 mm*10 um, Flow Rate: 80 (ml/min), water (0.05% ammonia hydroxide v/v) —CH₃CN, 25% CH₃CN to 55% CH₃CN over 30 min) to give (R)-(2-chloro-4-(4-fluorophenyl)-6,7-dihydrothieno[3,2-c]pyridin-5(4H)-yl)(imidazo[1,2-a]pyridin-6-yl)methanone (3.36 g, 8.16 mmol, 68% yield, 96.88% ee) as an off-white solid. LCMS: RT1=2.871 min, m/z 412.1 [M+H]⁺. ¹H NMR: (CDCl₃, 400 MHz) δ 8.25 (s, 1H), 7.64 (s, 1H), 7.59-7.55 (m, 2H), 7.27 (s, 1H), 7.06 (d, J=8.0 Hz, 1H), 6.98 (t, J=8.4 Hz, 2H), 6.68 (br, 1H), 6.49 (s, 1H), 3.90 (br, 1H), 3.32-3.31 (m, 1H), 2.94-2.88 (m, 1H), 2.74-2.69 (m, 1H).

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I): 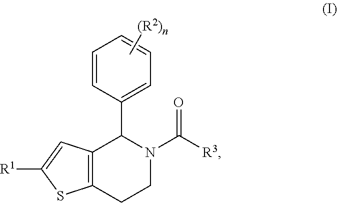
or a pharmaceutically acceptable salt thereof, wherein:
R¹ is halogen or optionally substituted alkyl;
each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};
n is 1, 2, 3, 4, or 5; and
R³ is optionally substituted, monocyclic or bicyclic carbocyclyl; optionally substituted, monocyclic or bicyclic aryl; optionally substituted, monocyclic or bicyclic heterocyclyl; or optionally substituted, monocyclic or bicyclic heteroaryl;
each instance of R^{2a} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group;
each instance of R^{2b} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or optionally, two R^{2b} are taken together with the intervening atoms to form optionally substituted heterocyclyl or heteroaryl; and
each instance of R^{2c} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

2. The compound of claim 1, wherein R¹ is halogen.

3. (canceled)

4. The compound of claim 1, wherein the compound of Formula (I) is of Formula (I-a): 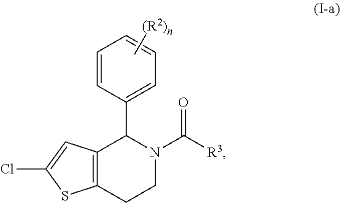
or a pharmaceutically acceptable salt thereof.

5. The compound of claim 1, wherein at least one instance of R² is halogen.

6. The compound of claim 5, wherein at least one instance of R² is —F.

7-11. (canceled)

12. A compound of Formula (II): 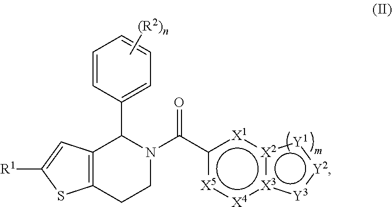
or pharmaceutically acceptable salt thereof, wherein:
R¹ is hydrogen, halogen, or optionally substituted alkyl;
each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};
n is 1, 2, 3, 4, or 5;
m is 1 or 2;
each instance of X¹, X², X³, X⁴, X⁵, Y¹, Y², and Y³ is independently C, CR^{C}, N, NR^{N}, S, or O, as valency permits; provided that at least one instance of X¹, X², X³, X⁴, or X⁵ is N, NR^{N}, S, or O;
each instance of R^{C} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};
each instance of R^{N} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group;
each instance of R^{2a} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group;
each instance of R^{2b} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or optionally, two R^{2b} are taken together with the intervening atoms to form optionally substituted heterocyclyl or heteroaryl;
each instance of R^{2c} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group.

13. The compound of claim 12, wherein at least one instance of X¹, X², X³, X⁴, or X⁵ is N.

14-19. (canceled)

20. The compound of claim 12, wherein the compound of Formula (II) is of Formula (II-a): 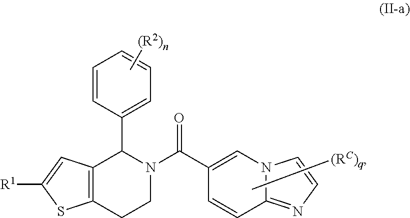
or a pharmaceutically acceptable salt thereof, wherein:
q is 0, 1, 2, 3, 4, or 5.

21. The compound of claim 12, wherein R¹ is halogen.

22-29. (canceled)

30. The compound of claim 12, wherein the compound of Formula (II) is of one of the following formulae: 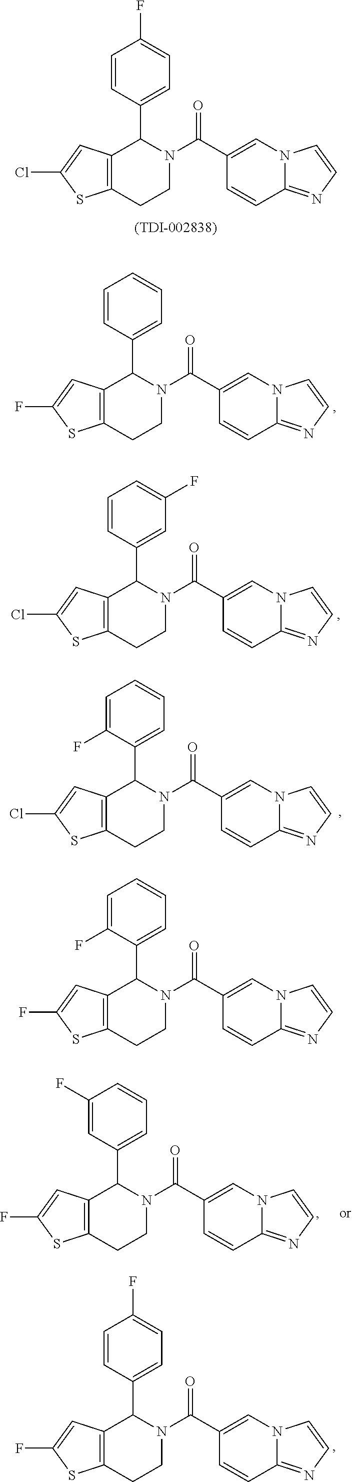
or a pharmaceutically acceptable salt thereof.

31. The compound of claim 30, wherein the compound is selected from the group consisting of: 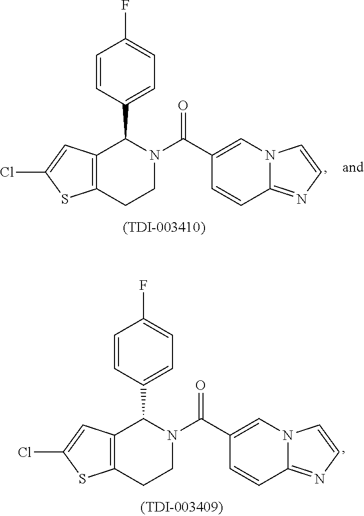 [SMILES: O=C(c1ccc2nccn2c1)N1CCc2sc(Cl)cc2[C@H]1c1ccc(F)cc1 ; O=C(c1ccc2nccn2c1)N1CCc2sc(Cl)cc2[C@@H]1c1ccc(F)cc1]
and pharmaceutically acceptable salts thereof.

32. (canceled)

33. A compound of Formula (III): 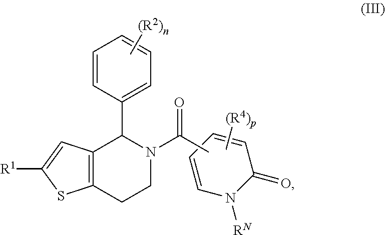
or pharmaceutically acceptable salt thereof, wherein:
R¹ is hydrogen, halogen, or optionally substituted alkyl;
each instance of R² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —OR^{2a}, —N(R^{2b})₂, or —SR^{2c};
each instance of R⁴ is independently hydrogen, —CN, —NO₂, —N₃, optionally substituted alkyl, optionally substituted carbocyclyl, optionally substituted aryl, optionally substituted heterocyclyl, optionally substituted five-membered heteroaryl, optionally substituted acyl, —OR^{2a}, —N(R^{2b})₂, or —SR^{2c};
each instance of R^{2a} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or an oxygen protecting group;
each instance of R^{2b} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a nitrogen protecting group; or optionally, two R^{2b} are taken together with the intervening atoms to form optionally substituted heterocyclyl or heteroaryl;
each instance of R^{2c} is independently hydrogen, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, or a sulfur protecting group;
n is 1, 2, 3, 4, or 5;
p is 1, 2, or 3; and
R^{N} is hydrogen, optionally substituted alkyl, optionally substituted acyl, or a nitrogen protecting group.

34. The compound of claim 33, wherein R¹ is hydrogen.

35-38. (canceled)

39. The compound of claim 33, wherein the compound of Formula (III) is of one of the following formulae: 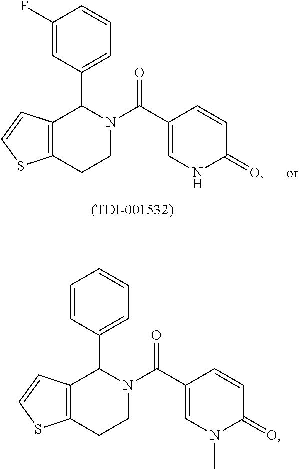 [SMILES: O=C(c1ccc(=O)[nH]c1)N1CCc2sccc2C1c1cccc(F)c1 ; Cn1cc(C(=O)N2CCc3sccc3C2c2ccccc2)ccc1=O]
or a pharmaceutically acceptable salt thereof.

40. The compound of claim 39, wherein the compound is selected from the group consisting of: 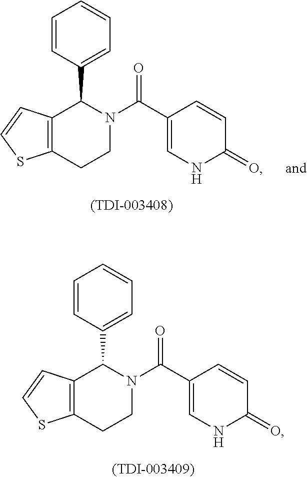 [SMILES: O=C(c1ccc(=O)[nH]c1)N1CCc2sccc2[C@H]1c1ccccc1 ; O=C(c1ccc(=O)[nH]c1)N1CCc2sccc2[C@@H]1c1ccccc1]
and pharmaceutically acceptable salts thereof.

41. A pharmaceutical composition comprising a compound of claim 1, or a pharmaceutically acceptable salt thereof, and a pharmaceutically acceptable excipient.

42-43. (canceled)

44. A method of treating a disease in a subject, the method comprising administering to the subject a therapeutically effective amount of a compound of claim 1, or a pharmaceutically acceptable salt thereof.

45. The method of claim 44, wherein the disease is a proliferative disease.

46-62. (canceled)

63. A method of inhibiting Hedgehog acyltransferase (Hhat), the method comprising contacting Hhat with a compound of claim 1, or a pharmaceutically acceptable salt thereof.

64-65. (canceled)

66. A method of inducing apoptosis, the method comprising contacting a cell with a compound of claim 1, or a pharmaceutically acceptable salt thereof.

67-73. (canceled)
